(19) United States
(12) Patent Application Publication
(10) Pub. No.: US 2009/0062788 A1
(43) Pub. Date: Mar. 5, 2009

(54) Electrical ablation surgical instruments

(76) Inventors: Gary L. Long, Cincinnati, OH (US); Omer J. Vakharia, Cincinnati, OH (US); David N. Plescia, Cincinnati, OH (US)

Correspondence Address: K&L GATES LLP, 535 SMITHFIELD STREET, PITTSBURGH, PA, 15222, US

(21) Appl. No.: 11/897,676

(22) Filed: Aug. 31, 2007

(51) Int. Cl. A61B 18/14

(52) U.S. Cl. 606/41

(57) ABSTRACT

A surgical instrument includes an ablation device. The ablation device includes an elongated flexible member having a proximal end and a distal end. The flexible member includes first and second lumens. A first needle electrode is configured to slideably move within the first lumen. A second needle electrode is located within the second lumen. The first and second needle electrodes are adapted to couple to an electrical waveform generator and to receive an electrical waveform sufficient to electrically ablate tissue located between the first and second needle electrodes.

### BACKGROUND

Electrical therapy techniques have been employed in medicine to treat pain and other conditions. Electrical ablation techniques have been employed in medicine to remove diseased tissue or abnormal growths, such as cancers or tumors, from the body. Electrical therapy probes comprising electrodes are employed to electrically treat diseased tissue at the tissue treatment region or target site. These electrical therapy probes comprising electrodes are usually inserted into the tissue treatment region percutaneously. There is a need for laparoscopic devices and techniques that provide minimally invasive access to the tissue treatment region or anatomic location, such as lung and liver tissue, for example, to diagnose and treat the condition more accurately and effectively. There is a need for such improved laparoscopic devices and techniques that are adapted to be introduced into the tissue treatment region through a trocar to electrically ablate or destroy the diseased tissue from the tissue treatment region.

### SUMMARY

In one general aspect, the various embodiments are directed to an ablation device. In one embodiment, the ablation device comprises an elongated flexible member having a proximal end and a distal end. The flexible member comprises first and second lumens. A first needle electrode is configured to slideably move within the first lumen. A second needle electrode is located within the second lumen. The first and second needle electrodes are adapted to couple to an electrical waveform generator and to receive an electrical waveform sufficient to electrically ablate tissue located between the first and second needle electrodes.

### FIGURES

The novel features of the various embodiments are set forth with particularity in the appended claims. The various embodiments, however, both as to organization and methods of operation, together with further objects and advantages thereof, may best be understood by reference to the following description, taken in conjunction with the accompanying drawings as follows.

FIG. 1 illustrates one embodiment of an electrical ablation system.

FIGS. 2A-D illustrate one embodiment of the electrical ablation device of the electrical ablation system shown in FIG. 1 in various phases of deployment.

FIG. 3 illustrates the use of one embodiment of the electrical ablation system to treat diseased tissue located on the surface of the liver.

FIGS. 4-10 illustrate one embodiment of an electrical ablation device.

FIG. 4 is a perspective side view of one embodiment of an electrical ablation device.

FIG. 5 is a side view of one embodiment of the electrical ablation device shown in FIG. 4.

FIG. 6 is a cross sectional perspective view of one embodiment of the electrical ablation device taken across line 6-6 in FIG. 4.

FIG. 7 is a cross-sectional perspective view of one embodiment of the electrical ablation device taken across line 7-7 in FIG. 4.

FIG. 8 is a front view of one embodiment of the electrical ablation device taken along line 8-8 in FIG. 5.

FIG. 9 is a back view of one embodiment of the electrical ablation device taken along line 9-9 in FIG. 5.

FIG. 10 is a cross-sectional view of one embodiment of the electrical ablation device taken along the longitudinal axis.

FIG. 11 illustrates the use of one embodiment of the electrical ablation system shown in FIGS. 4-10.

FIGS. 12-18 illustrate one embodiment of an electrical ablation device.

FIG. 12 is a top side perspective side view of the electrical ablation device.

FIG. 13 is a bottom side perspective view of one embodiment of the electrical ablation device shown in FIG. 12.

FIG. 14 is a side view of one embodiment of the electrical ablation device shown in FIG. 12.

FIG. 15 is a front view of one embodiment of the electrical ablation device taken along line 15-15 in FIG. 14.

FIG. 16 is a cross-sectional view of one embodiment of the electrical ablation device taken along the longitudinal axis.

FIG. 17 is a perspective view of one embodiment of the electrical ablation device and a handle assembly coupled to thereto.

FIG. 18 is a cross sectional view of one embodiment of the right hand portion of the handle assembly.

FIG. 19 illustrates one embodiment of an electrical ablation device.

FIG. 20 is an end view of one embodiment of the electrical ablation device shown in FIG. 19 taken along line 20-20.

FIG. 21 illustrates one embodiment of the electrical ablation device shown in FIG. 19 implanted in a blood vessel of a patient.

FIG. 22 illustrates one embodiment of the electrical ablation device shown in FIG. 19 located external to a patient.

FIG. 23 illustrates one embodiment of an electrical ablation device to treat diseased tissue within a lactiferous duct of a breast by delivering electrical energy to the lactiferous duct.

FIG. 24 illustrates one embodiment of an electrical ablation device to treat diseased tissue within a lactiferous duct of a breast by delivering electrical energy to the lactiferous duct.

FIG. 25 illustrates one embodiment of an electrical ablation device to treat diseased tissue located outside of a lactiferous duct of a breast by delivering electrical energy to the breast outside of the lactiferous duct.

FIG. 26 illustrates one embodiment of an electrical ablation device to treat diseased within a body cavity or organ by delivering electrical energy to the body cavity or organ.

FIGS. 27, 28, and 29 illustrate one embodiment of an electrical ablation device to treat diseased tissue within a body lumen using electrical energy.

FIG. 27 illustrates a sectioned view of one embodiment of an electrical ablation probe.

FIG. 28 illustrates an end view of one embodiment of the electrical ablation probe shown in FIG. 27.

FIG. 29 is a cross-sectional view of one embodiment of the electrical ablation probe that may be inserted in a lumen within a vessel.

FIG. 30 illustrates one embodiment of an electrical ablation device to treat diseased tissue within a breast by delivering electrical energy to a space defined within the breast.

### DESCRIPTION

The various embodiments described herein are directed to electrical therapy ablation devices. The electrical therapy ablation devices comprise probes and electrodes that can be positioned in or in proximity to a tissue treatment region (e.g., target site) within a patient either endoscopically or transcutaneously (percutaneously), and in some embodiments a combination thereof. An electrode may be introduced in the tissue treatment region (e.g., tissue treatment region) through a trocar. Other electrodes may be introduced in the tissue treatment region transcutaneously or percutaneously. The electrodes comprise an electrically conductive portion with a sharp point to facilitate insertion through the skin of a patient and to enhance local current density in the tissue treatment region during the treatment. Other electrodes may be introduced in the tissue treatment region by way of a natural orifice through a cannula or catheter. The placement and location of the electrodes can be important for effective and efficient therapy. Once positioned, the electrical therapy electrodes are adapted to deliver electrical current to the treatment region. The electrical current is generated by a control unit or generator located external to the patient. The electrical current may be characterized by a particular waveform in terms of frequency, amplitude, and pulse width. Depending on the diagnostic or therapeutic treatment rendered, the probes may comprise one electrode containing both a cathode and an anode or may contain a plurality of electrodes with at least one serving as a cathode and at least one serving as an anode.

Electrical therapy ablation may employ electroporation or electropermeabilization techniques where an externally applied electric field (electric potential) significantly increases the electrical conductivity and permeability of a cell plasma membrane. Electroporation is the generation of a destabilizing electric potential across such biological membranes. In electroporation, pores are formed when the voltage across the cell plasma membrane exceeds its dielectric strength. Electroporation destabilizing electric potentials are generally in the range of several hundred volts across a distance of several millimeters. Below certain magnitude thresholds, the electric potentials may be applied across a biological membrane as a way of introducing some substance into a cell, such as loading it with a molecular probe, a drug that can change the function of the cell, a piece of coding DNA, or increasing the uptake of drugs in cells. If the strength of the applied electrical field and/or duration of exposure to it are suitably chosen, the pores formed by the electrical pulse reseal after a short period of time, during such period extra-cellular compounds may enter into the cell. Below a certain field threshold, the process is reversible and the potential does not permanently damage the cell membrane. This process may be referred to as reversible electroporation (RE).

On the other hand, excessive exposure of live cells to large electric fields can cause apoptosis and/or necrosis—the processes that result in cell death. Excessive exposure of live cells to large excessive electrical fields or potentials across the cell membranes causes the cells to die and therefore may be referred to as irreversible electroporation (IRE).

Electroporation may be performed with devices called electroporators. These appliances create the electric current and send it through the cell. Electroporators may comprise two or more metallic (e.g., aluminum) electrically conductive electrodes connected to an energy source. The energy source generates an electric field having a suitable characteristic waveform output in terms of frequency, amplitude, and pulse width.

Endoscopy refers to looking inside the human body for medical reasons. Endoscopy may be performed using an instrument called an endoscope. Endoscopy is a minimally invasive diagnostic medical procedure used to evaluate the interior surfaces of an organ by inserting a small tube into the body, often, but not necessarily, through a natural body opening or through a relatively small incision. Through the endoscope, an operator may observe surface conditions of the organs including abnormal or diseased tissue such as lesions and other surface conditions. The endoscope may have a rigid or a flexible tube and in addition to providing an image for visual inspection and photography, the endoscope may be adapted and configured for taking biopsies, retrieving foreign objects, and introducing medical instruments to a tissue treatment region referred to as the target site. Endoscopy is a vehicle for minimally invasive surgery.

Laparoscopic surgery, is a minimally invasive surgical technique in which operations in the abdomen are performed through small incisions (usually 0.5-1.5 cm), keyholes, as compared to larger incisions needed in traditional surgical procedures. Laparoscopic surgery includes operations within the abdominal or pelvic cavities, whereas keyhole surgery performed on the thoracic or chest cavity is called thoracoscopic surgery. Laparoscopic and thoracoscopic surgery belong to the broader field of endoscopy.

A key element in laparoscopic surgery is the use of a laparoscope: a telescopic rod lens system, that is usually connected to a video camera (single chip or three chip). Also attached is a fiber optic cable system connected to a “cold” light source (halogen or xenon), to illuminate the operative field, inserted through a 5 mm or 10 mm cannula to view the operative field. The abdomen is usually insufflated with carbon dioxide gas to create a working and viewing space. The abdomen is essentially blown up like a balloon (insufflated), elevating the abdominal wall above the internal organs like a dome. Carbon dioxide gas is used because it is common to the human body and can be removed by the respiratory system if it is absorbed through tissue.

The embodiments of the electrical therapy ablation devices and techniques described herein may be employed to treat diseased tissue, tissue masses, tissue tumors, and lesions (diseased tissue) at a tissue treatment region (target site) within the body. The embodiments of the electrical therapy ablation devices and techniques described herein may be adapted to provide minimally invasive access to the tissue treatment region or anatomic location, such as lung and liver tissue, for example, to diagnose and treat the condition at the tissue treatment region more accurately and effectively. Such minimally invasive devices may be introduced into the tissue treatment region using a trocar. Once located at the target site, the diseased tissue is electrically ablated or destroyed. Some portions of the electrical therapy ablation devices may be inserted into the tissue treatment region percutaneously. Other portions of the electrical therapy ablation devices may be introduced in the tissue treatment region endoscopically (e.g., laparoscopically and/or thoracoscopically) or through small incisions. The electrical therapy ablation devices may be employed to deliver energy to the diseased tissue to ablate or destroy tumors, masses, lesions, and other abnormal tissue growths. In one embodiment, the electrical therapy ablation devices and techniques described herein may be employed in the treatment of cancer by quickly creating necrosis and destroying live cancerous tissue in-vivo. Minimally invasive therapeutic procedures to treat diseased tissue by introducing medical instruments to a tissue treatment region through a natural opening of the patient are known as Natural Orifice Translumenal Endoscopic Surgery (NOTES)™.

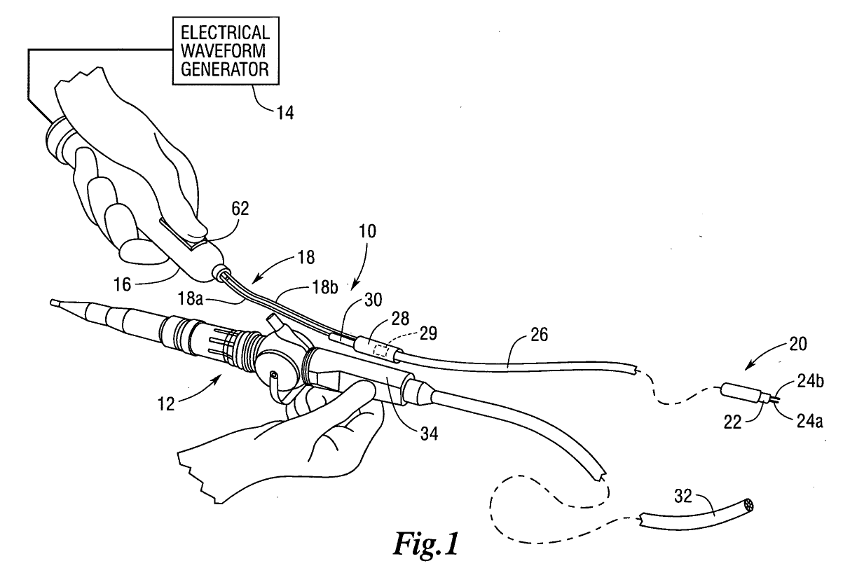

FIG. 1 illustrates one embodiment of an electrical ablation system 10. The electrical ablation system 10 may be employed to treat diseased tissue such as tumors and lesions inside a patient with electrical energy. The electrical ablation system 10 may be used to treat the desired tissue treatment region in endoscopic, laparoscopic, thoracoscopic, or open surgical procedures via small incisions or keyholes as well as external and non-invasive medical procedures. The electrical ablation system 10 may be configured to be positioned within a natural opening of the patient such as the colon or the esophagus and can be passed through the natural opening to reach the tissue treatment region or target site. The electrical ablation system 10 also may be configured to be positioned through a small incision or keyhole on the patient and can be passed through the incision to reach a tissue treatment region or target site through a trocar. The tissue treatment region may be located in the esophagus, colon, liver, breast, brain, lung, and other organs or locations within the body. The electrical ablation system 10 can be configured to treat a number of lesions and ostepathologies comprising metastatic lesions, tumors, fractures, infected site, inflamed sites, and the like. Once positioned in the tissue treatment region, the electrical ablation system 10 can be configured to treat and ablate the diseased tissue in that region. In one embodiment, the electrical ablation system 10 may be adapted to treat diseased tissue, such as cancers, of the gastrointestinal (GI) tract, esophagus, or lung that may be accessed orally. In another embodiment, the electrical ablation system 10 may be adapted to treat diseased tissue, such as cancers, of the liver or other organs that may be accessible trans-anally through the colon and/or the abdomen via well known procedures.

In one embodiment, the electrical ablation system 10 may be employed in conjunction with a flexible endoscope 12 (also referred to as endoscope 12), such as the GIF-100 model available from Olympus Corporation. In one embodiment, the flexible endoscope 12, laparoscope, or thoracoscope may be introduced into the patient trans-anally through the colon, the abdomen via an incision or keyhole and a trocar, or through the esophagus. The endoscope 12 or laparoscope assists the surgeon to guide and position the electrical ablation system 10 near the tissue treatment region to treat diseased tissue on organs such as the liver. In another embodiment, the flexible endoscope 12 or thoracoscope may be introduced into the patient orally through the esophagus to assist the surgeon guide and position the electrical ablation system 10 near the tissue treatment region to treat diseased tissue near the gastrointestinal (GI) tract, esophagus, or lung.

In the embodiment illustrated in FIG. 1, the flexible endoscope 12 comprises an endoscope handle 34 and an elongate relatively flexible shaft 32. The distal end of the flexible shaft 32 of the flexible endoscope 12 may comprise a light source a viewing port, and an optional working channel. The viewing port transmits an image within its field of view to an optical device such as a charge coupled device (CCD) camera within the flexible endoscope 12 so that an operator may view the image on a display monitor (not shown).

The electrical ablation system 10 generally comprises an electrical ablation device 20, a plurality of electrical conductors 18, a handpiece 16 comprising an activation switch 62, and an electrical waveform generator 14 coupled to the activation switch 62 and the electrical ablation device 20. The electrical ablation device 20 comprises a relatively flexible member or shaft 22 that may be introduced to the tissue treatment region through a trocar.

One or more needle electrodes, such as first and second electrical therapy needle electrodes 24a,b, extend out from the distal end of the electrical ablation device 20. In one embodiment, the first needle electrode 24a is the negative electrode and the second needle electrode 24b is the positive electrode. The first needle electrode 24a is electrically connected to a lead such as a first electrical conductor 18a and is coupled to the negative terminal of the electrical waveform generator 14. The second needle electrode 24b is electrically connected to a lead such as a second electrical conductor 18b and is coupled to the positive terminal of the electrical waveform generator 14. Once located in the tissue treatment region, the needle electrodes 24a,b deliver electrical energy of a predetermined characteristic shape, amplitude, frequency, and duration as supplied by the electrical waveform generator 14.

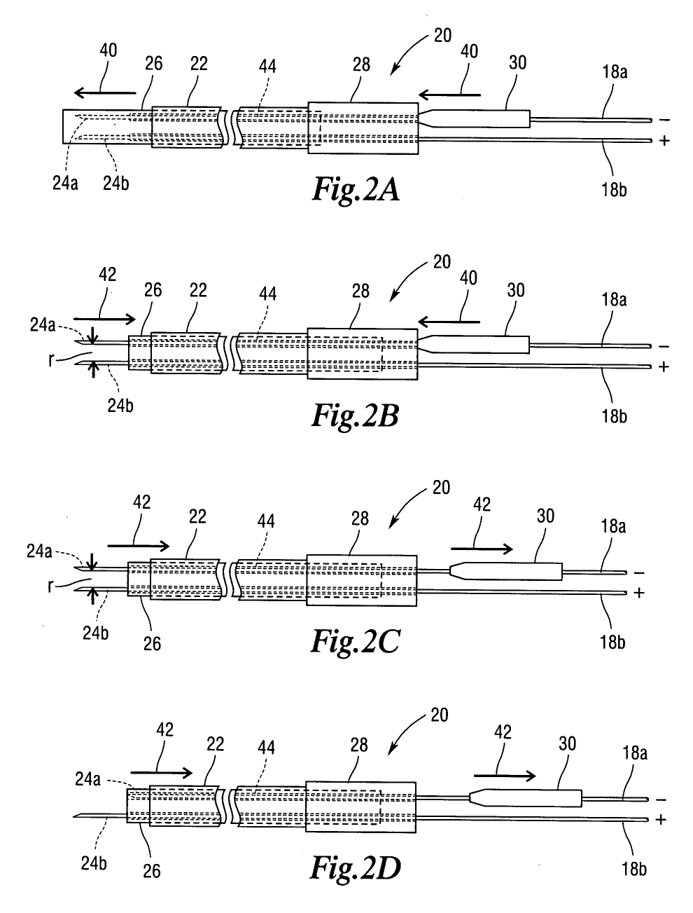

A protective sleeve or sheath 26 is slidably disposed over the flexible shaft 22 and within a handle 28 portion. The sheath 26 is slideable and may be located over the needle electrodes 24a,b to protect the trocar when the electrical ablation device 20 is pushed therethrough. Either one or both of the needle electrodes may be adapted and configured in the electrical ablation device 20 to slideably move in and out of a cannula or lumen formed within a flexible shaft 22. In the illustrated embodiments, the first needle electrode 24a, the negative electrode, can be slideably moved in and out of the distal end of the flexible shaft 22 using a slide member 30 to retract and/or advance the first needle electrode 24a. The second needle electrode 24b, the positive electrode, is fixed in place. The second needle electrode 24b provides a pivot about which the first needle electrode 24a can be moved in an arc to other points in the tissue treatment region to treat large portions of diseased tissue that cannot be treated by fixing the first and second needle electrodes 24a,b in one location. The first and second electrical conductors 18a,b are provided through a handle 28 portion. The first electrical conductor 18a, which is coupled to the first needle electrode 24a, is coupled to the slide member 30. The slide member 30 is employed to advance and retract the first needle electrode 24a, which is slidably movable within a lumen formed within the flexible shaft 22. This is described in more detail in FIGS. 2A-D.

The electrical ablation device 20 may be introduced to the desired tissue treatment region in endoscopic, laparoscopic, thoracoscopic, or open surgical procedures as well as external and non-invasive medical procedures. Once the first and second needle electrodes 24a,b are located at respective first and second positions in the tissue treatment region, manual operation of the switch 62 of the handpiece 16 electrically connects or disconnects the needle electrodes 24a,b to the electrical waveform generator 14. Alternatively, the switch 62 may be mounted on, for example, a foot switch (not shown). The needle electrodes 24a,b may be referred to herein as endoscopic or laparoscopic electrodes. As previously discussed, either one or both of the needle electrodes 24a,b may be adapted and configured in the electrical ablation device 20 to slideably move in and out of a cannula or lumen formed within a flexible shaft 22.

In various other embodiments, transducers or sensors 29 may be located in the handle 28 portion of the electrical ablation device 20 to sense the force with which the needle electrodes 24a,b penetrate the tissue in the tissue treatment zone. This feedback information may be useful to determine whether the either one or both of the needle electrodes 24a,b have been inserted in a diseased tissue region. As is well known, cancerous tumors tend to be denser than healthy tissue and thus would require greater force to insert the needle electrodes 24a,b therein. The operator, surgeon, or clinician can physically sense when the needle electrodes 24a,b are placed within the tumor tissue in the tissue treatment zone. If the transducers or sensors 29 are employed, the information may be processed and displayed by circuits located either internally or externally to the electrical waveform generator 14. The sensor 29 readings may be employed to determine whether the needle electrodes 24a,b have been properly located in the tumor tissue thereby assuring that a suitable margin of error has been achieved in locating the needle electrodes 24a,b.

In one embodiment, the first and second needle electrodes 24a,b are adapted to receive electrical energy from a generator. The electrical energy conducted through the first and second needle electrodes 24a,b forms an electrical field at a distal end of the first and second needle electrodes 24a,b that is suitable to treat diseased tissue. In one embodiment, the electrical waveform generator 14 delivers the energy to generate the electrical field. The waveform generator 14 may be configured to generate electrical fields at a predetermined frequency, amplitude, polarity, and pulse width suitable to destroy diseased tissue cells. Application of the electrical field to the cell membranes destroys the diseased tissue located in a tissue treatment region by a process referred to as electrical ablation. The electrical waveform generator 14 may be configured to generate electrical fields in the form of direct current (DC) electrical pulses having a predetermined frequency, amplitude, and pulse width suitable to destroy cells in diseased tissues. The polarity of the DC pulses may be either positive or negative relative to a reference electrode. The polarity of the DC pulses may be reversed or inverted from positive-to-negative or from negative-to-positive any predetermined number of times to destroy the diseased tissue cells. For example, the DC electrical pulses may be delivered at a frequency in the range of 1-20 Hz, amplitude in the range of ±100 to ±1000VDC, and pulse width in the range of 0.01-100 ms, for example. As an illustrative example, electrical waveforms having amplitude of +500VDC and pulse duration of 20 ms may be delivered at a pulse repetition rate or frequency of 10 Hz to destroy a reasonably large volume of diseased tissue. In one embodiment, the DC polarity of the electrical pulses may be reversed by the electrical waveform generator 14. The embodiments, however, are not limited in this context.

In one embodiment, the first and second needle electrodes 24a,b are adapted to receive electrical fields in the form of an IRE waveform from an IRE generator. In another embodiment, the first and second needle electrodes 24a,b are adapted to receive a radio frequency (RF) waveform from an RF generator. In one embodiment, the electrical waveform generator 14 may be a conventional, bipolar/monopolar electrosurgical IRE generator such as one of many models commercially available, including Model Number ECM 830, available from BTX Molecular Delivery Systems Boston, Mass. The IRE generator generates electrical waveforms having predetermined frequency, amplitude, and pulse width. The application of these electrical waveforms to the cell membranes of the diseased tissue causes the diseased cells to die. Thus, the IRE electrical waveforms may be applied to the cell membranes of diseased tissue in the tissue treatment region in order to kill the diseased cells and ablate the diseased tissue. IRE electrical waveforms suitable to destroy the cells of diseased tissues are generally in the form of DC electrical pulses delivered at a frequency in the range of 1-20 Hz, amplitude in the range of +100 to +1000VDC, and pulse width in the range of 0.01-100 ms. For example, an electrical waveform having amplitude of +500VDC and pulse duration of 20 ms may be delivered at a pulse repetition rate or frequency of 10 HZ to destroy a reasonably large volume of diseased tissue. Unlike RF ablation systems which require high powers and energy input into the tissue to heat and destroy, IRE requires very little energy input into the tissue, rather the destruction of the tissue is caused by high electric fields. It has been determined that in order to destroy living tissue, the electrical waveforms have to generate an electric field of at least 30,000V/m in the tissue treatment region.

The polarity of the electrodes 24a,b may be switched electronically to reverse the polarity of the cell. Unlike conventional IRE, reversing the polarity of the electrodes 24a,b may reduce the muscular contractions due to a constant electric field generated in the tissue. Accordingly, in one embodiment, the polarity of the electrical pulses may be inverted or reversed by the electrical waveform generator 14. For example, the electrical pulses initially delivered at a frequency in the range of 1-20 Hz and amplitude in the range of +100 to +1000VDC, and pulse width in the range of 0.01-100 ms. The polarity of the electrical pulses then may be reversed such that the pulses have amplitude in the range of −100 to −1000VDC. For example, an electrical waveform comprising DC pulses having amplitude of +500VDC may be initially applied to the treatment region or target site and after a predetermined period, the amplitude of the DC pulses may be reversed to −500VDC. As previously discussed, to destroy a reasonably large volume of diseased tissue, the pulse duration may be 20 ms and may be delivered at a pulse repetition rate or frequency of 10HZ. The embodiments, however, are not limited in this context.

In one embodiment, the electrical waveform generator 14 may comprise a RF waveform generator. The RF generator may be a conventional, bipolar/monopolar electrosurgical generator such as one of many models commercially available, including Model Number ICC 350, available from Erbe, GmbH. Either a bipolar mode or monopolar mode may be used. When using the bipolar mode with two electrodes, one electrode is electrically connected to one bipolar polarity, and the other electrode is electrically connected to the opposite bipolar polarity. If more than two electrodes are used, the polarity of the electrodes may be alternated so that any two adjacent electrodes have opposite polarities. Either the bipolar mode or the monopolar mode may be used with the illustrated embodiment of the electrical ablation system 10. When using the bipolar mode with two needle electrodes 24a,b the first needle electrode 24a may be electrically connected to one bipolar polarity, and the second needle electrode 24b may be electrically connected to the opposite bipolar polarity (or vice-versa). If more than two electrodes are used, the polarity of the needle electrodes 24a,b is alternated so that any two adjacent electrodes have opposite polarities.

In either case, the electrical (e.g., the IRE or RF) waveform generator 14, when using the monopolar mode with two or more electrodes, a grounding pad is not needed on the patient. Because a generator will typically be constructed to operate upon sensing connection of ground pad to the patient when in monopolar mode, it can be useful to provide an impedance circuit to simulate the connection of a ground pad to the patient. Accordingly, when the electrical ablation system 10 is used in monopolar mode without a grounding pad, an impedance circuit can be assembled by one skilled in the art, and electrically connected in series with either one of the needle electrodes 24a,b that would otherwise be used with a grounding pad attached to a patient during monopolar electrosurgery. Use of an impedance circuit allows use of the IRE generator in monopolar mode without use of a grounding pad attached to the patient.

FIGS. 2A-D illustrate one embodiment of the electrical ablation device 20 of the electrical ablation system 10 shown in FIG. 1 in various phases of deployment. FIG. 2A illustrates an initial phase of deployment wherein the sheath 26 is extended in the direction indicated by arrow 40 to cover the needle electrodes 24a,b. As shown in FIG. 2A, the electrical ablation device 20 is ready to be introduced into the tissue treatment region through a trocar, for example. FIG. 2B illustrates another phase of deployment wherein the sheath 26 is retracted within the handle 28 in the direction indicated by arrow 42. In this phase of deployment the first and second needle electrodes 24a,b extend through the distal end of the flexible shaft 22 and are ready to be inserted into the tissue in the tissue treatment region. The first needle electrode 24a may be retracted in direction 42 through a lumen 44 formed in the flexible shaft 22 by holding the handle 28 and pulling on the slide member 30. FIG. 2C illustrates a transition phase wherein the first needle electrode 24a is the process of being retracted in the direction indicated by arrow 42 by pulling on the slide member 30 handle in the same direction. FIG. 2D illustrates another phase of deployment wherein the first needle electrode 24a is in a fully retracted position. In this phase of deployment the electrical ablation device 20 can be pivotally rotated about an axis 46 defined by the second needle electrode 24b. Once the electrical ablation device 20 is rotated in an arc about the pivot formed by the second needle electrode 24b, the first needle electrode 24a may be located in a new location in the tissue treatment region within a radius “r” defined as the distance between the first and second needle electrodes 24a,b. The needle electrode 24a,b can be located in a plurality of positions in and around the tissue treatment region to be able to treat a much larger tissue treatment region. The first and second needle electrodes 24a,b are spaced apart by a distance “r”. Spacing the first and second needle electrodes 24a,b further apart allows the electrodes to treat a larger diseased tissue region and generate an electric field over a much larger tissue treatment region. In this manner, the operator can treat a larger tissue treatment region of a cancerous lesion, a polyp, or a tumor, for example. Retracting the first needle electrode 24a and pivoting about the second needle electrode 24b enables the surgeon or clinician to target and treat a larger tissue treatment region essentially comprising a circular region having a radius “r”, which is the distance between the first and second needle electrodes 24a,b.

The operator, surgeon, or clinician may employ the endoscope 12 comprising at least a light source and a viewing port located at a distal end thereof to assist in visually locating the target diseased tissue region using endoscopic visualization feedback by employing. The needle electrodes 24a,b are energized by the electrical waveform generator 14 to deliver an IRE or an RF electrical waveform that is suitable to treat the specific diseased tissue located between the first and second needle electrodes 24a,b. Locating the needle electrodes 24a,b in the tissue treatment region independently provides the operator flexibility in positioning the needle electrodes 24a,b relative to the tissue treatment region.

The electrical conductors 18a,b are electrically insulated from each other and surrounding structures, except for the electrical connections the respective needle electrodes 24a,b. The distal end of flexible shaft 22 is proximal to the first and second needle electrodes 24a,b within the field of view of the flexible endoscope 12 thus enabling the operator to see the tissue treatment region to be treated near the first and second needle electrodes 24a,b. This technique provides a more accurate way to locate the first and second needle electrodes 24a,b in the tissue treatment region.

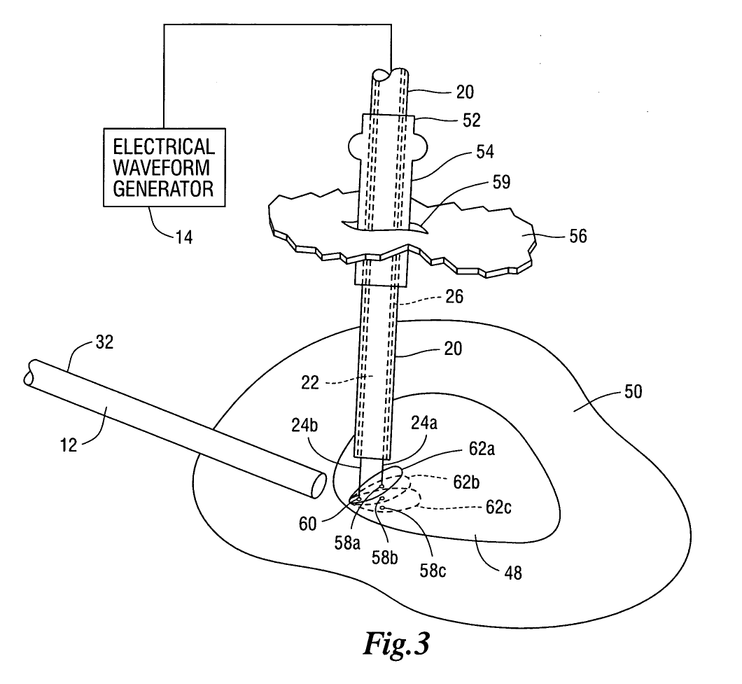

FIG. 3 illustrates the use of one embodiment of the electrical ablation system 10 to treat diseased tissue 48 located on the surface of the liver 50. In use, the electrical ablation device 20 may be introduced into the tissue treatment region through a port 52 of a trocar 54. The trocar 54 is introduced into the patient via a small incision 59 formed in the skin 56. The endoscope 12 may be introduced into the patient trans-anally through the colon or through a small incision or keyhole in the abdomen. The endoscope 12 is employed to guide and locate the distal end of the electrical ablation device 20 near the diseased tissue 48 otherwise referred to as the target site. Prior to introducing the flexible shaft 22 through the trocar 54, the sheath 26 is slid over the flexible shaft 22 in a direction toward the distal end thereof to cover the needle electrodes 24a,b (as shown in FIG. 2A) until the distal end of the electrical ablation device 20 reaches the diseased tissue 48 region. Once the electrical ablation device 20 has been fully introduced into the diseased tissue 48 region, the sheath 26 is retracted to expose the needle electrodes 24a,b (as shown in FIG. 2B) to treat the diseased tissue 48. The operator positions the first needle electrode 24a at a first position 58a and the second needle electrode 24b at a second position 60 using endoscopic visualization such that the diseased tissue 48 to be treated lies within the field of view of the flexible endoscope 12. The operator may locate the first needle electrode 24a located in the first position 58a near a perimeter edge of the diseased tissue 48. Once the needle electrodes 24a,b are located in the tissue treatment region and they are energized, a first necrotic zone 62a is created. For example, when the first and second needle electrodes 24a,b are placed in the desired location at positions 60 and 58a, the first and second needle electrodes 24a,b may be energized by an electrical field supplied by the electrical waveform generator 14 suitable to destroy the diseased tissue 48 in the first necrotic zone 62a. As previously discussed, the electrical field may be in the form of an IRE or RF waveform, or any electrical waveform suitable to treat the diseased tissue cells at the target site. For example, in an IRE embodiment, the first and second needle electrodes 24a,b may be energized with an electrical waveform having amplitude of approximately 500VDC and a pulse width of approximately 20 ms at a frequency of approximately 10 Hz. In this manner, the diseased tissue 48 in the first necrotic zone 62a may be destroyed. The size of the necrotic zone is substantially dependent on the size and separation of the needle electrodes 24a,b. The treatment time is defined as the time that the needle electrodes 24a,b are activated or energized to destroy the diseased tissue. The treatment time is relatively short and may be approximately 1 or 2 seconds. Therefore, in a relatively short time, the surgeon or clinician can rapidly treat a larger treatment zone (e.g., create a larger necrotic zone) by repositioning or relocating the first needle electrode 24a within the diseased tissue region 48.

This procedure may be repeated to destroy relatively larger portions of the diseased tissue 48. The position 60 is a pivot point about which the first needle electrode 24a may be rotated in an arc of radius “r”, which is the distance between the first and second electrodes 24a,b. Prior to rotating about the second needle electrode 24b, the first needle electrode 24a is retracted by pulling on the slide member 30 (FIGS. 1 and 2A-D) in a direction toward the proximal end and rotating the electrical ablation device 20 about the pivot point formed at position 60 by the second needle electrode 24b. Once the first needle electrode 24a is rotated to a second position 58b, it is advanced to engage the diseased tissue at point 58b by pushing on the slide member 30 in a direction towards the distal end. A second necrotic zone 62b is formed upon energizing the first and second electrodes 24a,b in the new location. A third necrotic zone 62c is formed by retracting the first needle electrode 24a, pivoting about pivot point 60 and rotating the first needle electrode 24a to a new location, advancing the first needle electrode 24a into the diseased tissue 48 and energizing the first and second electrodes 24a,b. This process may be repeated as often as necessary to create any number of necrotic zones 62n within multiple circular areas of radius “r”, for example, that is suitable to destroy the entire diseased tissue 48 region, where n is any positive integer. At anytime, the surgeon or clinician can reposition both the first and second needle electrodes 24a,b and begin the process anew. Those skilled in the art will appreciate that similar techniques may be employed to treat any other diseased tissues accessed trans-anally through the colon and/or the abdomen and/or accessed orally through the esophagus or the stomach. Therefore, the embodiments are not limited in this context.

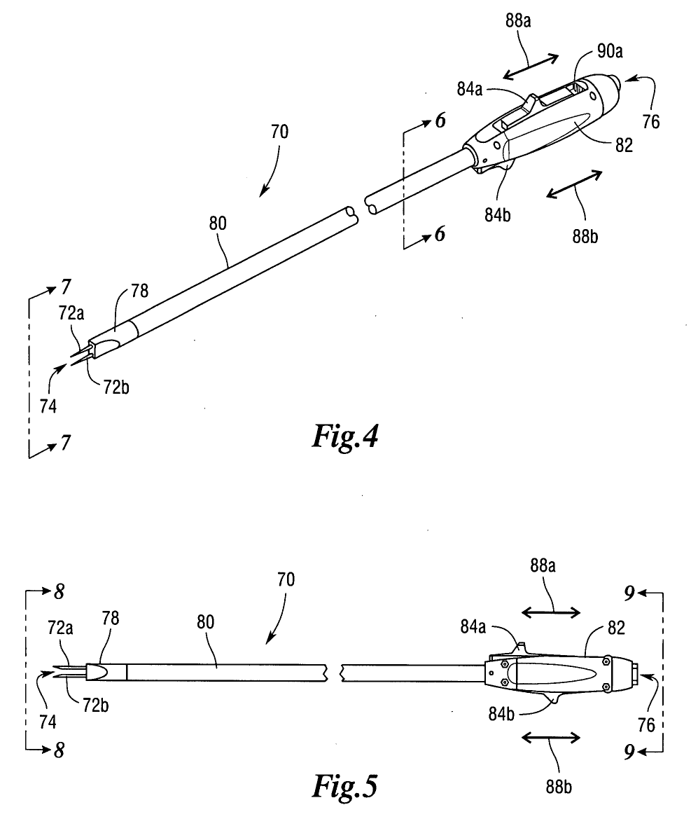

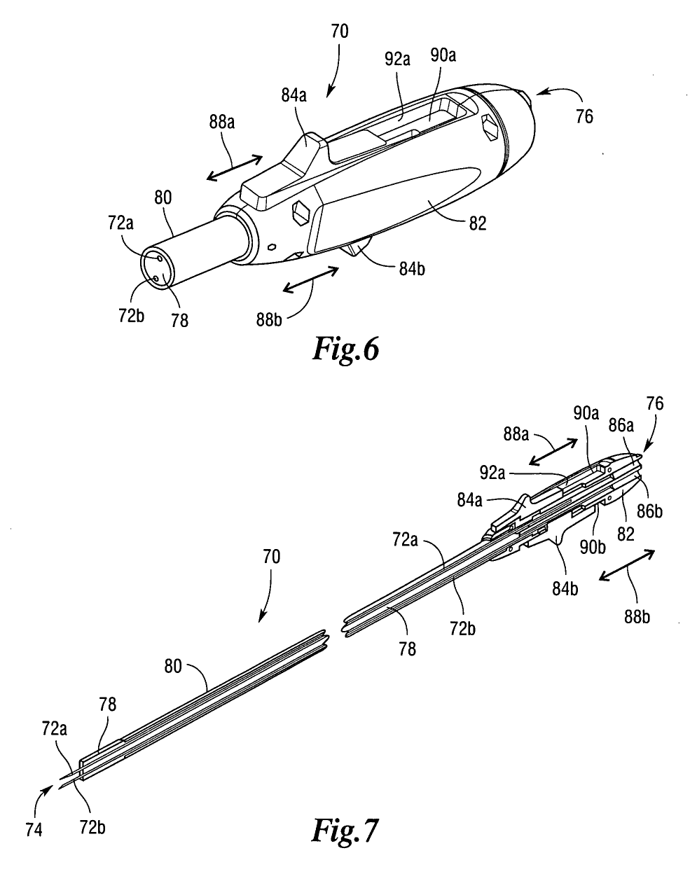

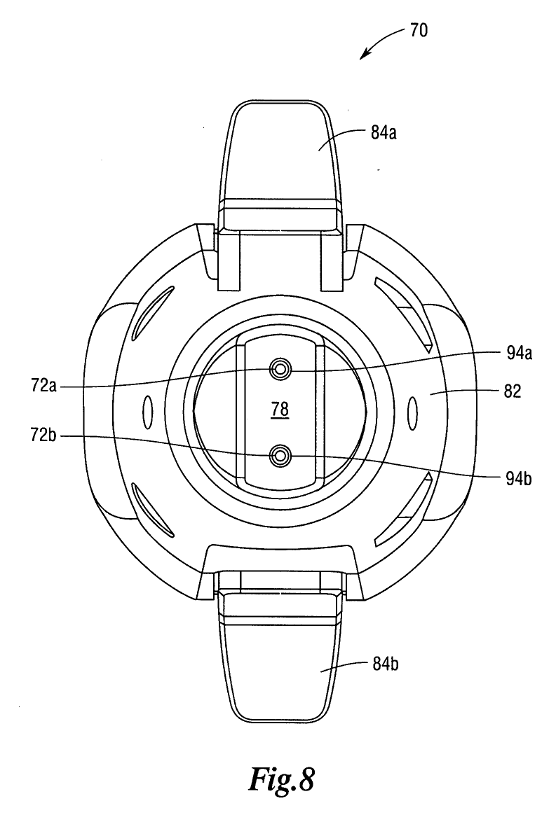

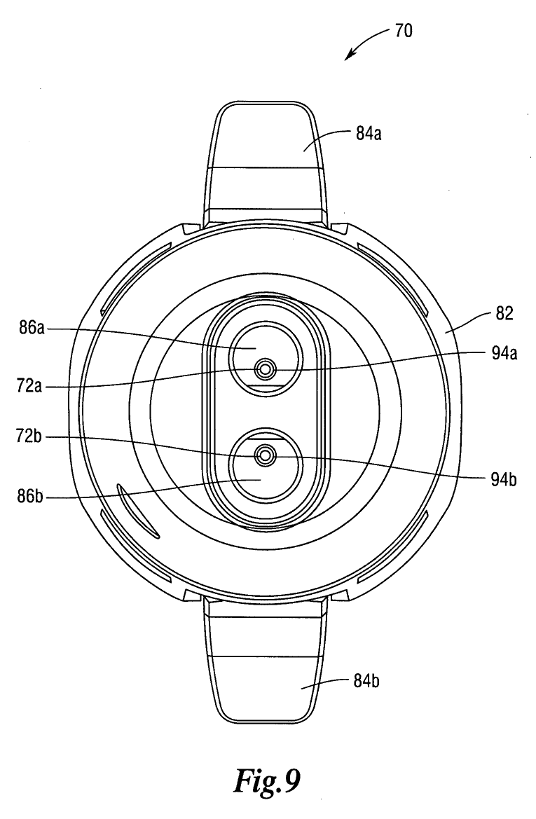

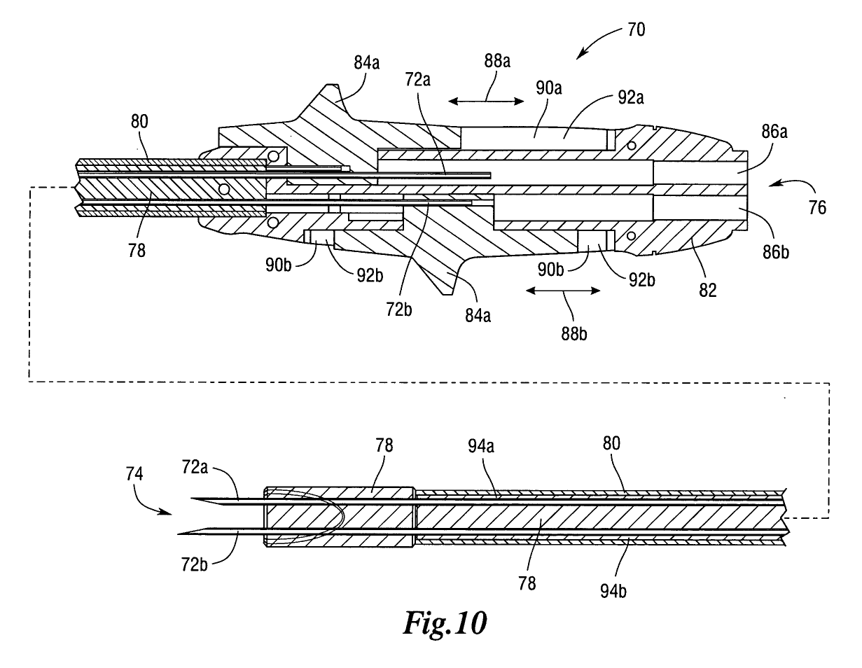

FIGS. 4-10 illustrate one embodiment of an electrical ablation device 70. FIG. 4 is a perspective side view of one embodiment of the electrical ablation device 70. FIG. 5 is a side view of one embodiment of the electrical ablation device 70. FIG. 6 is a cross sectional perspective view of one embodiment of the electrical ablation device 70 taken across line 6-6 in FIG. 4. FIG. 7 is cross-sectional perspective view of one embodiment of the electrical ablation device 70 taken across line 7-7 in FIG. 4. FIG. 8 is a front view of one embodiment of the electrical ablation device 70 taken along line 8-8 in FIG. 5. FIG. 9 is a back view of the electrical ablation device 70 taken along line 9-9 in FIG. 5. FIG. 10 is a cross-sectional view of one embodiment of the electrical ablation device 70 taken along the longitudinal axis.

In one embodiment, the electrical ablation device 70 may be employed to treat diseased tissue at a target tissue site in a patient. The embodiment illustrated in FIGS. 4-10 may be adapted to treat colorectal cancer (e.g., colon cancer) using electrical fields such as, for example, IRE, although the embodiments are not limited in this context as the electrical ablation device 70 can be adapted and/or configured to treat a variety of diseased tissues in the esophagus, liver, breast, brain, lung, and other organs employing a variety of electrical energy fields and waveforms. Colorectal cancer, also called colon cancer or bowel cancer, includes cancerous growths in the colon, rectum, and appendix. It is the third most common form of cancer and the second leading cause of death among cancers in the western world. Many colorectal cancers are thought to arise from adenomatous polyps in the colon. These mushroom-like growths are usually benign, but some may develop into cancer over time. The majority of the time, the diagnosis of localized colon cancer is through colonoscopy. Therapy is usually through surgery, which in many cases is followed by chemotherapy. It would be desirable to have a substantially simple and effective technique to destroy cancerous tissue in the colon. As previously described, any suitable electrical energy fields or waveforms such as IRE techniques, for example, may be employed to effectively destroy cancerous tissue cells. As previously discussed, in one embodiment, the polarity of the electrical pulses may be inverted or reversed by the electrical waveform generator 14 during the treatment process.

With reference now to FIGS. 4-10, the electrical ablation device 70 comprises a elongated flexible shaft 78 that houses two needle electrodes 72a,b. The needle electrodes 72a,b are free to extend past the distal end 74 of the electrical ablation device 70. In one embodiment, the first and second needle electrodes 72a,b are adapted to receive an electrical field such as an IRE waveform, for example, from an IRE generator. In another embodiment, the first and second needle electrodes 72a,b are adapted to receive an RF waveform from an RF generator. In one embodiment, the first and second needle electrodes 72a,b are connected to the respective positive and negative outputs of a high-voltage DC generator (e.g., the electrical waveform generator 14) at the proximal end 76. The needle electrodes 72a,b supply high voltage DC pulses to the tissue treatment region to destroy the cancerous cells located at the target site. Electrical conductors carrying the high voltage DC pulses from the electrical waveform generator 14 (FIG. 1) may be coupled to the needle electrodes 72a,b through openings 86a,b forming electrical receptacles at the proximal end 76 to receive conductive elements coupled to the electrical waveform generator 14. As previously discussed, in one embodiment, the polarity of the electrical pulses may be inverted or reversed by the electrical waveform generator 14 during the treatment process.

The electrical ablation device 70 may be employed in a method of treatment cancerous tissue without destroying red blood cells. Red blood cells (erythrocytes) are not destroyed in the same manner as bi-layer lipid cells (cancerous cells). In one embodiment, the electrical ablation device 70 may be introduced through an existing endoscope, such as the endoscope 12 shown in FIG. 1. The cancerous tissue region may be visually located with the endoscope 12 and therapy may be applied by extending the needle electrodes 72a,b into the diseased tissue and energizing the needle electrodes 72a,b. Typically, 20 to 40 pulses of approximately 500-700 volts DC at approximately 100-400 Us duration each are sufficient to destroy cancerous tissues.

The flexible shaft 78 comprises first and second lumen 94a,b formed therein to slidably receive the respective first and second needle electrodes 72a,b. A flexible sheath 80 extends longitudinally from a handle portion 82 to the distal end 74. The handle portion 82 comprises a first slide member 84a and a second slide member 84b. The slider members 84a,b are received in respective slots 90a and 90b (FIG. 7) defining respective wall 92a,b. The slider members 84a,b are coupled to the respective first and second needle electrodes 72a,b. The first slide member 84a is movable in direction 88a and the second slider is movable in direction 88b. Accordingly, moving the first slide member 84a in direction 88a toward the proximal end 76 retracts the first needle electrode 72a into the flexible shaft 78. Similarly, moving the second slide member 84b in direction 88b toward the proximal end 76 retracts the second needle electrode 72b into the flexible shaft 78. The first and second needle electrodes 72a,b are independently movable by way of the respective first and second slider members 84a,b. To deploy the first and second needle electrodes 72a,b the respective first and second slider members 84a,b can be moved independently in respective directions 88a,b toward the distal end 74.

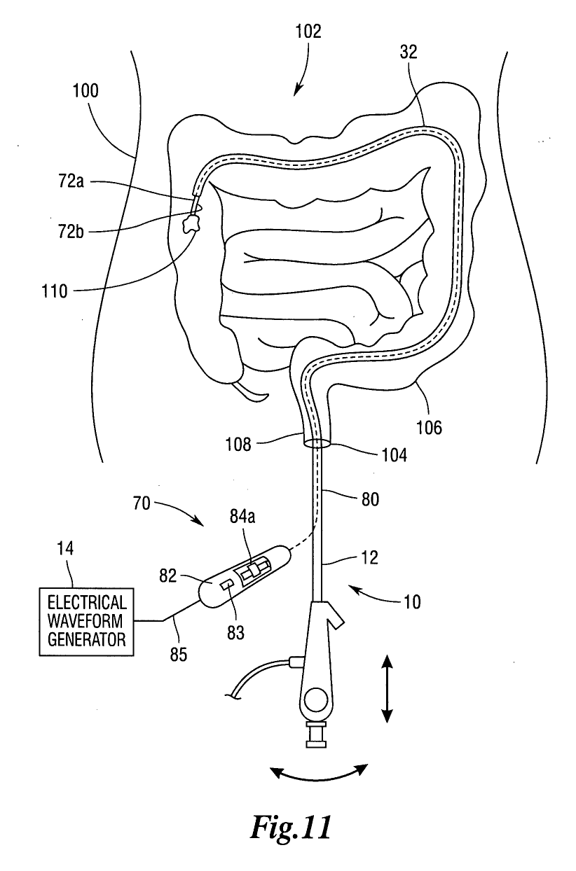

FIG. 11 illustrates the use of one embodiment of the electrical ablation system 70 shown in FIGS. 4-10. The electrical ablation device 70 is inserted into a hollow body or natural opening of a patient 100. The electrical ablation device 70 is introduced to diseased tissue 110 through the colon 102. The electrical ablation device 70 is inserted into the colon 102 through the anus 104. The colon 102 includes a sphincter muscle 106 disposed between the anus 104 and the rectum 108. The electrical ablation system 70 is steerable and maneuverable and may be steered or maneuvered through several turns through the colon 102.

The electrical ablation system 70 may be introduced endoscopically through the endoscope 12. The operator inserts the flexible shaft 32 of the endoscope 12 into the anus 104 and maneuvers it through the colon 102. The operator uses endoscopic visualization through the viewing port of the endoscope 12 to position the distal end 74 of the electrical ablation device 70 at the target site of the diseased tissue 110. At the target site, the first and second needle electrodes 72a,b are inserted into the diseased tissue 110 such that they are placed in intimate contact with the diseased tissue 110 to be treated within the field of view of the flexible endoscope 12. Watching through the viewing port of the endoscope 12, the operator can actuate a switch 83 located on the handle 82 to electrically connect the electrodes 72a,b to the waveform generator 14 through a corresponding set of conductors 85 inserted through the electrical receptacle openings 86a,b. Electric current then passes through the portion of the diseased tissue 110 positioned between the electrodes 72a,b. When the operator observes that the tissue within the field of view has been sufficiently ablated, the operator deactuates the switch 83 to stop the ablation. The operator may reposition either of the endoscopic electrodes 72a for subsequent tissue treatment, or may withdraw the electrical ablation device 70 (together with the flexible endoscope 12). As previously discussed above with reference to FIGS. 1 and 2A-D, in the embodiment described in FIGS. 4-11, either one or both of the electrodes 72a,b may retracted with one of the electrodes acting as a pivot while the other electrode is repositioned to enable the operator to cover a larger area of the tissue treatment region.

If the diseased tissue 110 is located on the liver, the distal end of the endoscope 12 can be advanced into the sigmoid colon. Once in the sigmoid colon an instrument such as a needle knife can be advanced through the lumen of the endoscope 12. The needle knife can then cut an opening through the sigmoid colon and into the peritoneal space (under visualization). The endoscope 12 can then be advance into the peritoneal space and manipulate until the liver is in view. This can be done under visualization using the view from the endoscope 12 or with fluoroscopy. The electrical ablation device 70 and the first and second electrodes 72a,b are then advanced into the liver to the target site.

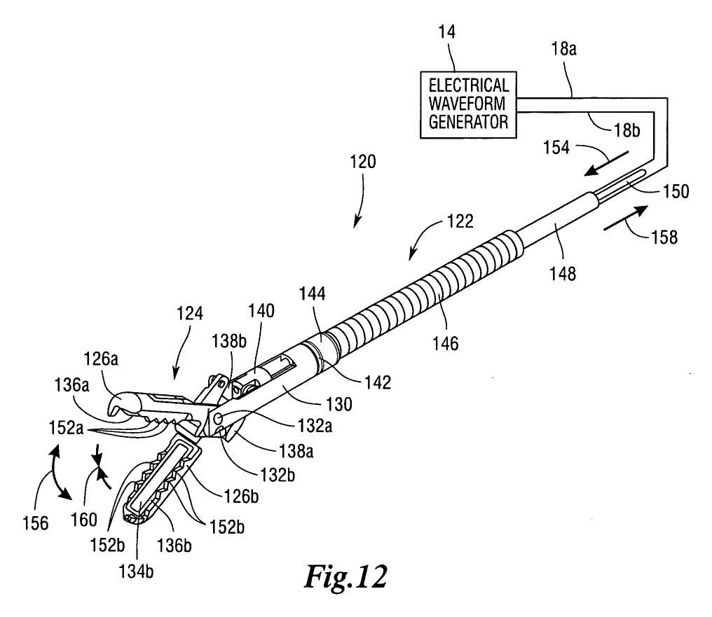

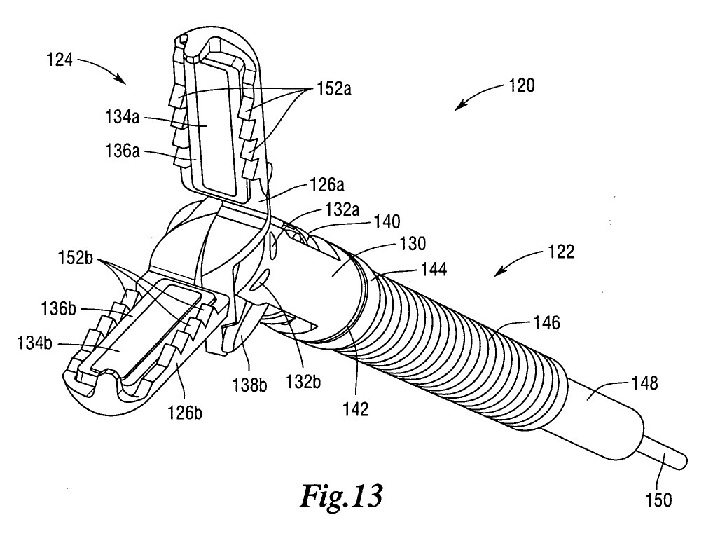

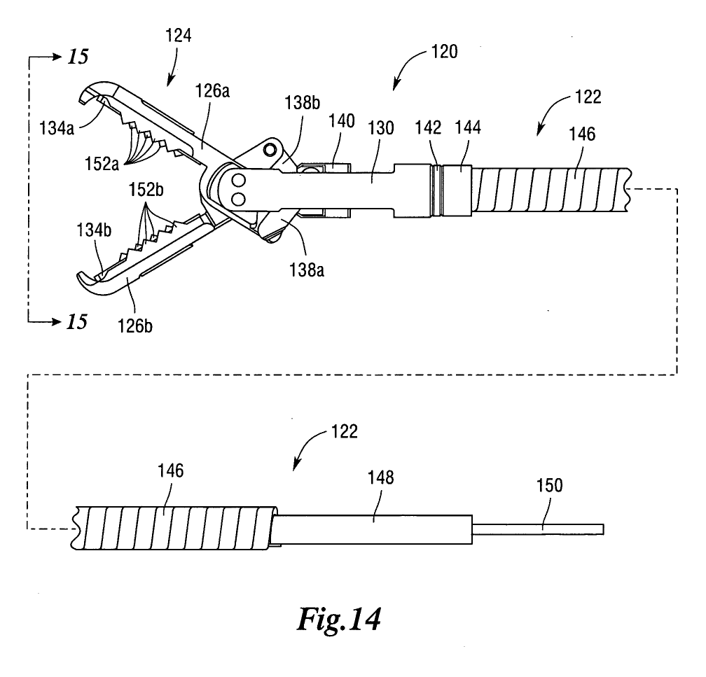

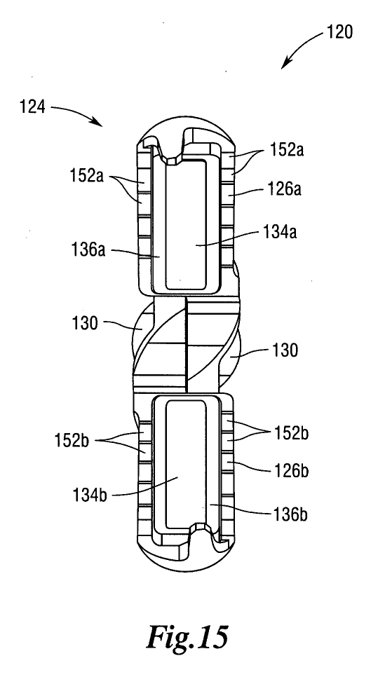

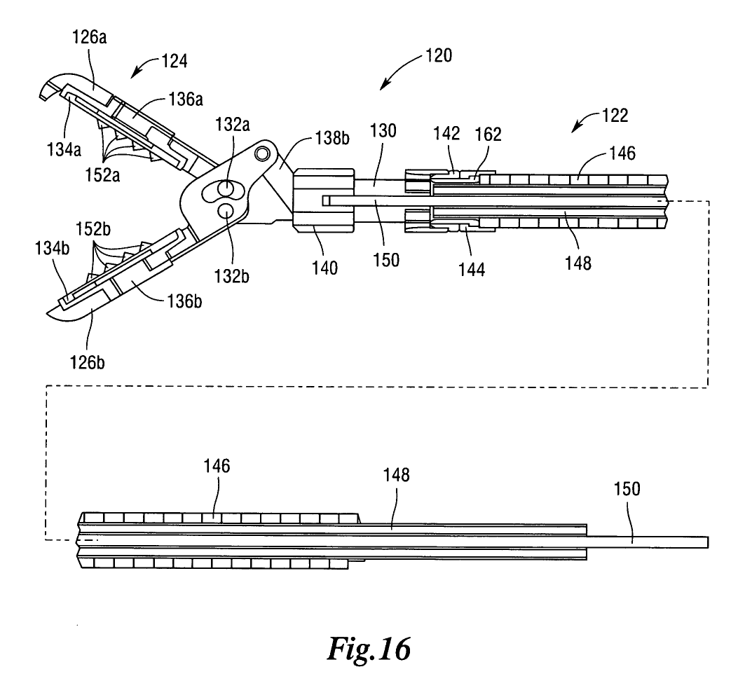

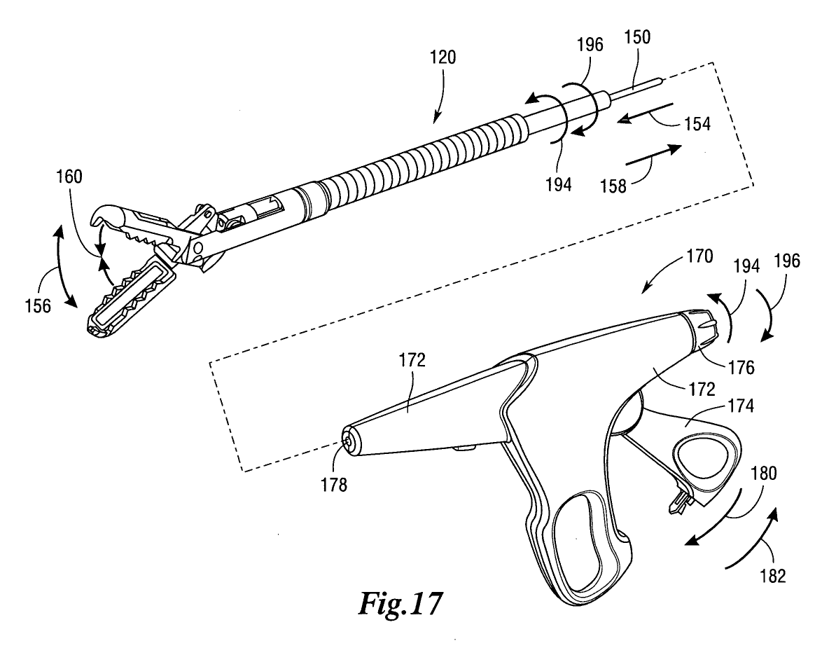

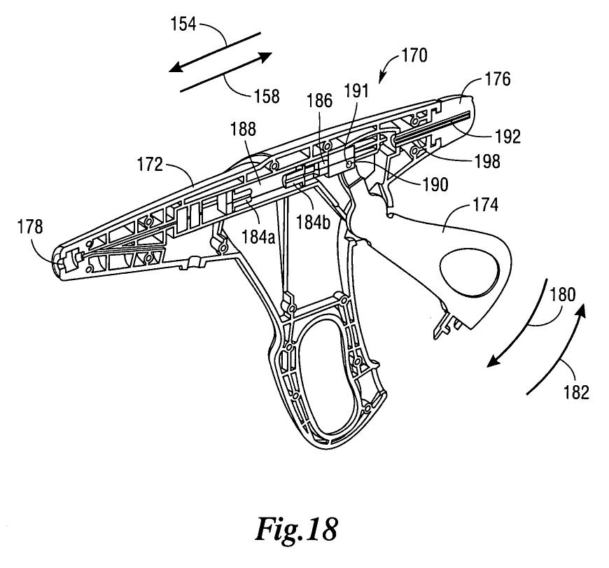

FIGS. 12-18 illustrate one embodiment of an electrical ablation device 120. FIG. 12 is a top side perspective side view of the electrical ablation device 120. FIG. 13 is a bottom side perspective view of one embodiment of the electrical ablation device 120. FIG. 14 is a side view of one embodiment of the electrical ablation device 120. FIG. 15 is a front view of one embodiment of the electrical ablation device taken along line 15-15 in FIG. 14. FIG. 16 is a cross-sectional view of one embodiment of the electrical ablation device 120 taken along the longitudinal axis. FIG. 17 is a perspective view of one embodiment of the electrical ablation device and a handle assembly coupled to thereto. FIG. 18 is a cross sectional view of one embodiment of the right hand portion of the handle assembly.

With reference now to FIGS. 12-16, the electrical ablation device 120 comprises an elongated flexible portion 122 and a clamp jaw portion 124. The clamp jaw portion 124 comprises a first jaw member 126a and a second jaw member 126b. The first and second jaw members 126a,b are pivotally coupled to a clevis 130 by respective first and second clevis pins 132a,b. The first jaw member 126a comprises an electrode portion 136a and an electrical insulator portion 136a. The first jaw member 126a also comprises a plurality of serrations 152a or teeth. The second jaw member 126b comprises an electrode portion 136b and an electrical insulator portion 136b. The second jaw member 126b also comprises a plurality of serrations 152b or teeth. The first jaw member 126a is coupled to an actuator 140 by a first link 138a. The second jaw member 126b is coupled to the actuator 140 by a second link 138b.

The elongated portion 122 comprises an elongated flexible member 146 coupled to the clevis 130 by a bushing coupler 142 and a ring capture 144. In one embodiment, the elongated flexible member 146 comprises a flat spring coil pipe. An inner housing coupler 162 (FIG. 16) coupled to the ring capture 144 and the bushing coupler 142. A multi-lumen elongated flexible member 148 is disposed within the elongated flexible member 146. An elongated actuator member 150 is provided within one of the lumens formed within the multi-lumen elongated flexible member 148. The elongated actuator member 150 may be formed as a solid rod or a tube. The elongated actuator member 150 is coupled to the actuator 140. The elongated actuator member 150 moves reciprocally in the directions indicated by arrows 154 and 158. When the elongated actuator member 150 is moved in the direction indicated by arrow 154, the first and second jaw members 126a,b open in the direction indicated by arrow 156. When the elongated actuator member 150 is moved in the direction indicated by arrow 158, the first and second jaw members 126a,b close in the direction indicated by arrow 160. Accordingly, the first and second jaw members 126a,b cooperate and act like forceps or tongs to grasp and contain tissue, such as dysplastic or cancerous mucosal tissue, for example, between the serrations 152a,b.

First and second electrical conductors 118a,b are electrically coupled to the respective first and second electrodes 134a,b formed in the respective first and second jaw members 126a,b. In one embodiment, the first and second electrodes 134a,b may be formed having a substantially flat paddle-like shape. The first and second electrical conductors 118a,b are received through lumens formed in the multi-lumen elongated flexible member 148 and are coupled to the first and second electrodes 134a,b in any suitable manner. A switch may be coupled to the electrical conductors 118a,b to enable an operator to activate and deactivate the first and second electrodes 134a,b after tissue at the desired target site is grasped between the first and second jaw members 126a,b.

In one embodiment, the electrical ablation device 120 may be employed to treat diseased tissue at a target tissue site in a patient. The embodiment illustrated in FIGS. 12-16 may be adapted to treat various types of diseased tissue such as dysplastic or cancerous mucosal tissue that can be found in the body. When such diseased mucosal tissue is discovered it may be biopsied and observed over time. Although, the diseased mucosal tissue may be removed or treated with a thermal device to destroy the tissue, removing the diseased mucosal tissue or destroying it in this manner can damage the thin wall thickness of the particular organ (such as esophagus or stomach) adjacent to the mucosal tissue to the extent that a perforation can occur in the organ. The embodiment of the electrical ablation device 120 shown in FIGS. 12-16 comprise a forceps or paddle-like device comprising the first and second jaw members 126a,b operatively coupled to the actuator 140 and the elongated actuator member 150 to grasp and contain the mucosal tissue between the first and second electrodes 134a,b. Once the tissue is grasped or engaged by the serrations 152a,b formed in the first and second jaw members 126a,b and contained between the first and second electrodes 134a,b, electrical energy may be applied to the first and second electrodes 134a,b to destroy the tissue contained therebetween. The first and second electrodes 134a,b comprise electrically conductive surfaces adapted to receive an electrical field from a suitable waveform generator. In one embodiment, the first and second electrodes 134a,b are adapted to receive an electrical field such as an IRE waveform from a suitable IRE waveform generator. In another embodiment, the first and second electrodes 134a,b are adapted to receive a RF waveform from a suitable RF waveform generator. In one embodiment, the first and second electrodes 134a,b are connected to the electrical waveform generator 14 such as a high voltage DC waveform generator (±500VDC), for example. It has been shown that when high electric fields are applied to tissue, the cell membrane will form an aqueous pathway through which molecules can flow (electroporation). If the electric field is increased to a sufficient level, the wall of the cell will rupture and subsequent apoptosis/necrosis will occur (irreversible electroporation). This occurs on the order of 1 millisecond, therefore very little energy is put into the tissue and very little heating occurs. Therefore, the tissue can be treated more precisely and safely with the electrical ablation device 120 than complete removal or thermal destruction of the diseased mucosal tissue. As previously discussed, in one embodiment, the polarity of the electrical pulses may be inverted or reversed by the electrical waveform generator 14 during the treatment process. Electrical waveform generators are discussed in commonly owned United States patent applications titled “Electroporation Apparatus, System, and Method”, Ser. No. 11/706,591 to Long and “Electroporation Ablation Apparatus, System, and Method”, Ser. No. 11/706,766 to Long, both of which are incorporated herein by reference.

FIG. 17 is a perspective view of the electrical ablation device 120 and a handle assembly 170 coupled to thereto. The handle assembly 170 comprises a base handle portion 172, a trigger 174, a rotation knob 176, and an opening 178 to receive the distal end of the elongated actuator member 150. The trigger 174 is operatively coupled to the elongated actuator member 150. When the trigger 174 is pivotally moved (e.g., squeezed) in the direction indicated by arrow 180, the elongated actuator member 150 moves in the direction indicated by arrow 158, and the first and second jaw members 126a,b close in the direction indicated by arrow 160. When the trigger 174 is pivotally moved (e.g., released) in the direction indicated by arrow 182, the elongated actuator member 150 moves in the direction indicated by arrow 154, and the first and second jaw members 126a,b open in the direction indicated by arrow 156. The distal end of the elongated actuator member 150 is received within a neck portion 198 (FIG. 18) of the rotation knob 176. When the rotation knob 176 is rotated in the direction indicated by arrow 194 the electrical ablation device 120 is also rotated in the direction indicated by arrow 194. When the rotation knob 176 is rotated in the direction indicated by arrow 196 the electrical ablation device 120 is also rotated in the direction indicated by arrow 196.

FIG. 18 is a sectional view of the right hand portion of the handle assembly 170. The distal end of the elongated actuator member 150 is received in the opening 178. The distal end of the elongated actuator member 150 is fixedly received in the first and second force limit spring holders 184a,b, shaft collar 186, and a slot 192 or groove formed in the neck portion 198 of the rotation knob 176. The trigger 174 is coupled to a force limit slider 188 at a pivot point 190 by a pivot pin 191. Accordingly, when the trigger 174 is squeezed in direction 180, the force limit slider 188 slides in the direction indicated by arrow 158 and a portion of the distal end of the elongated actuator member 150 is slideably received within the neck portion 198 of the rotation knob 176. When the trigger 174 is released, the force limit slider 188 moves in the direction indicated by arrow 154 by the spring force stored in the spring.

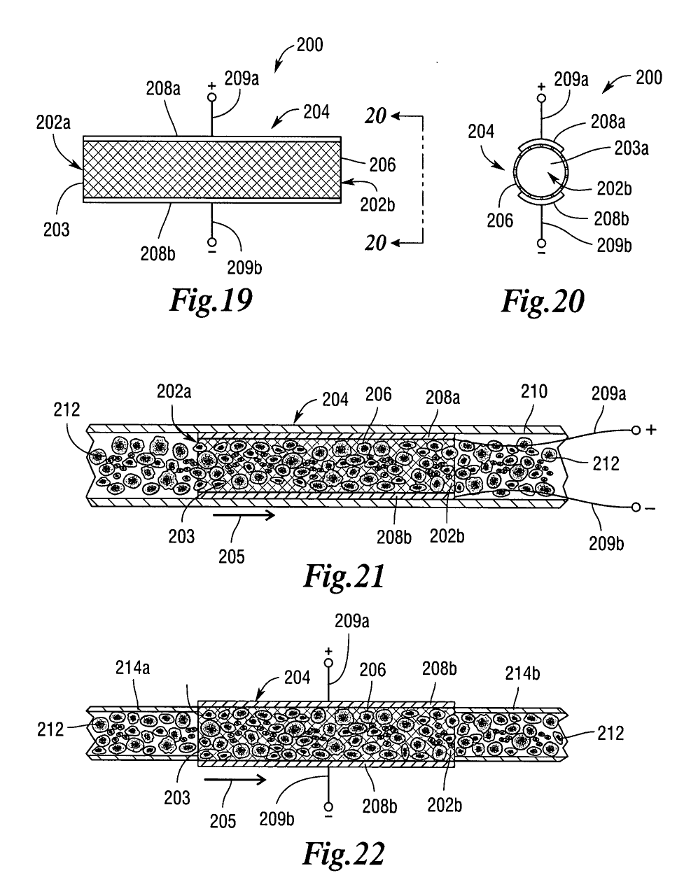

FIG. 19 illustrates one embodiment of an electrical ablation device 200. FIG. 20 is an end view of the electrical ablation device 200 taken along line 20-20. The electrical ablation device 200 can be employed to treat cancerous cells in a circulatory system of a patient. Cancerous cells can become free and circulate in the circulatory system as well as the lymphomic system. These cells can form metastasis in organs such as in the liver. In one embodiment, the electrical ablation device 200 employs an electrical field suitable to destroy tissue cells at the treatment site. The electrical ablation device 200 comprises a tubular member 204 defining a central opening 203 for receiving blood therethrough. In one embodiment, the tubular member 204 may be a small, expandable tube used for inserting in a vessel or other part, similar to a stent. The tubular member 204 may be temporarily implanted in the vessel for electrical ablation treatment of blood flowing therethrough. In another embodiment, the tubular member 204 may be located externally to the patient to receive blood from a blood vessel of a patient may be received from the patient, treated, and circulated back to the patient through a blood vessel after treatment. As previously discussed, in one embodiment, the polarity of the electrical pulses may be inverted or reversed by the electrical waveform generator 14 during the treatment process.

In the embodiment illustrated in FIGS. 19 and 20, blood is received through an opening 202a of the tubular member 204. The tubular member 204 comprises a small, expandable body 206 that defines a central opening 203 and may be inserted into a vessel or other body part via a slender thread, rod, or catheter. The tubular member 204 comprises a first positive electrode 208a and a second negative electrode 208b. The first and second electrodes 208a,b are coupled to the electrical waveform generator 14 (FIG. 1) via respective electrical conductors 209a,b. The first and second electrodes 208a,b may be located on opposite portions of the tubular member 204. In one embodiment, the first and second electrodes 208a,b are adapted to receive an IRE waveform from an IRE generator. In another embodiment, the first and second electrodes 208a,b are adapted to receive a RF waveform from an RF generator. In one embodiment, the electrical ablation device 200 employs IRE to destroy the cancerous cells without destroying healthy blood cells. IRE has been shown to be an effective way to destroy the cancerous cells. An IRE electric field is created between the first and second electrodes 208a,b when they are energized by the electrical waveform generator 14. The first and second electrodes 208a,b are adapted to receive high voltage DC pulses from the waveform generator 14 to destroy the cancerous cells in the bloodstream or other flowable substance passing through the tubular member 204. If the pulse width of the voltage is reduced to a sufficiently short length (t<60 nanoseconds) and the voltage is increased (V>10 kV/cm), then the contents (organelles) of the cancerous cells will be altered in a way that will cause the cell to become necrotic (apoptosis) yet the plasma membrane (cell wall) will not be affected. Likewise the plasma membrane of the red blood cell will be preserved and because red blood cells do not contain organelles similar to cancerous cells, they will not be destroyed.

FIG. 21 illustrates one embodiment of the electrical ablation device 200 implanted in a blood vessel 210 of a patient. The stent-like tubular member 204 may be implanted internally within the patient. The stent-like tubular member 204 may be inserted into a tubular structure, such as the blood vessel 210 to receive blood 212 through an inlet opening 202a. The blood 212 flows through the stent-like tubular member 204 in the direction indicated by arrow 205 and exits through an outlet opening 202b. When the electrodes 208a,b are energized with high voltage electrical energy such as DC pulses generated by the waveform generator 14 (FIG. 1), for example, the cancerous cells which pass through the central opening 203 are destroyed. As previously discussed, however, the red blood cells (erythrocytes) are not destroyed if a suitable pulse width and voltage is selected to treat the cancerous cells, the red blood cells will not be destroyed.

FIG. 22 illustrates one embodiment of the electrical ablation device 200 located external to a patient. In another embodiment, the tubular member 204 or may be located externally of the patient to circulate blood 212 therethrough to treat the cancerous cells in the blood 212 with IRE. The tubular member 204 receives the blood 212 in the inlet opening 202a from one end of a first blood vessel 214a of a patient and supplies the blood 212 to a second blood vessel 214b of the patient through an outlet opening 202b as the blood 212 flows in direction 205. As the blood 212 passes through the central opening 203, the cancerous cells are destroyed by the electrical field waveform while the normal red blood cells are unharmed.

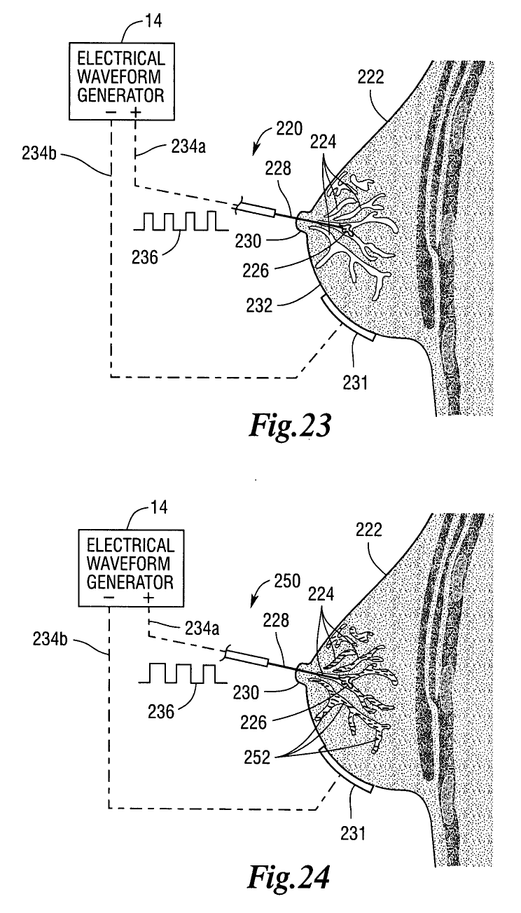

FIG. 23 illustrates one embodiment of an electrical ablation device 220 to treat diseased tissue within a lactiferous duct of a breast by delivering electrical energy to the lactiferous duct. FIG. 23 illustrates a cross-sectional view of a women's breast 222. In one embodiment, the electrical ablation device 220 may be employed to treat cancerous tissue 226 within lactiferous ducts 224 of the breast 222. Cancerous tissue 226 in the breast 222 including breast cancer tumors that are 2 cm or less have may be treated with ablation using electrical fields. These techniques destroy the cancerous tissue 226 in a less invasive manner as compared with lumpectomy or mastectomy. The electrical ablation device 220 employs electrical fields to destroy the cancerous tissue 226. As previously discussed, in one embodiment the electrical fields may be applied to destroy tissue cells at the treatment site. In one embodiment, the electrical ablation device 220 comprises a first electrode 228 comprising an electrically conductive elongated member such as a wire or a flexible electrically conductive tube. The first electrode 228 is introduced through a nipple 230 portion of the breast 222 into one of the lactiferous ducts 224 of the breast 222 where the cancerous tissue 226 is located. The first electrode 228 may be introduced into the lactiferous duct 224 under fluoroscopy, ultrasound guidance, or other well known techniques. A second electrode 231 comprising an electrically conductive pad is located on an exterior or outside portion 232 of the breast 222. The second electrode 231 has a much larger surface area that the first electrode 228. In one embodiment, the first and second electrodes 228, 231 are adapted to receive electrical fields in the form of an IRE waveform from an IRE generator. In another embodiment, the first and second electrodes 228, 231 are adapted to receive electrical fields in the form of a RF waveform from an RF generator. In the illustrated embodiment, the first electrode 228 is connected to the positive output of the waveform generator 14 through a first lead 234a and the second electrode 231 is connected to a negative output of the waveform generator 14 through a second lead 234b. As previously discussed, electrical waveform generator 14 is capable of generating high voltage pulse waveforms of various amplitude, frequency, and pulse duration. In other embodiments, the polarity of the first and second electrodes 228, 231 may be inverted. Multiple pulses may be supplied to the first electrode 228 and the pad of the second electrode 231 to destroy the cancerous tissue 226 occupying the space in the duct 224. A pulse train 236 comprising 20 to 40 pulses of ±500 to ±700 VDC of approximately 0.4 milliseconds in duration each is sufficient to destroy the cancerous tissue 226. As previously discussed, in one embodiment, the polarity of the electrical pulses may be inverted or reversed by the electrical waveform generator 14 during the treatment process.

FIG. 24 illustrates one embodiment of an electrical ablation device 250 to treat diseased tissue within a lactiferous duct of a breast by delivering electrical energy to the lactiferous duct. FIG. 24 illustrates a cross-sectional view of a women's breast 222. In one embodiment, a conductive fluid 252 may be introduced into the duct 224 to extend the operating range of the first electrode 228 to treat the cancerous tissue 226 within the duct 224. As discussed above, the pulse train 236 comprising 20 to 40 pulses of ±500 to ±700 VDC of approximately 0.4 milliseconds in duration each is sufficient to destroy the cancerous tissue 226. As previously discussed, in one embodiment, the polarity of the electrical pulses may be inverted or reversed by the electrical waveform generator 14 during the treatment process.

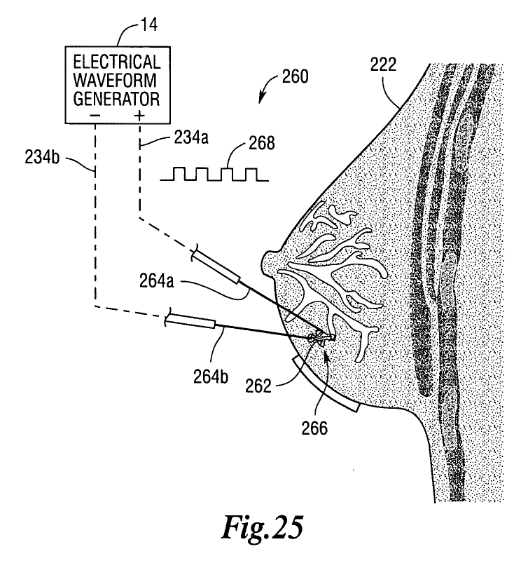

FIG. 25 illustrates one embodiment of an electrical ablation device 260 to treat diseased tissue located outside of a lactiferous duct of a breast by delivering electrical energy to the breast outside of the lactiferous duct. For example, the electrical ablation device 260 may be employed to treat breast cancer tissue 262 that is not located within a lactiferous duct 224 using electrical energy. FIG. 25 illustrates a cross-sectional view of a women's breast 222. To treat a cancerous tissue 262 of a nonductal tumor, first and second needle electrodes 264a,b are located into the tumor target site 266 directly. In one embodiment, the first and second electrodes 264a,b are adapted to receive an electrical field such as, for example, an IRE waveform from an IRE generator. In another embodiment, the first and second electrodes 264a,b are adapted to receive a RF) waveform from an RF generator. In one embodiment, IRE pulses may be applied to the target site 266 to destroy the cancerous tissue 262. A pulse train 268 comprising 20 to 40 pulses of ±500 to ±700 VDC of approximately 0.4 milliseconds in duration each is sufficient to destroy the cancerous tissue 226. As previously discussed, in one embodiment, the polarity of the electrical pulses may be inverted or reversed by the electrical waveform generator 14 during the treatment process.

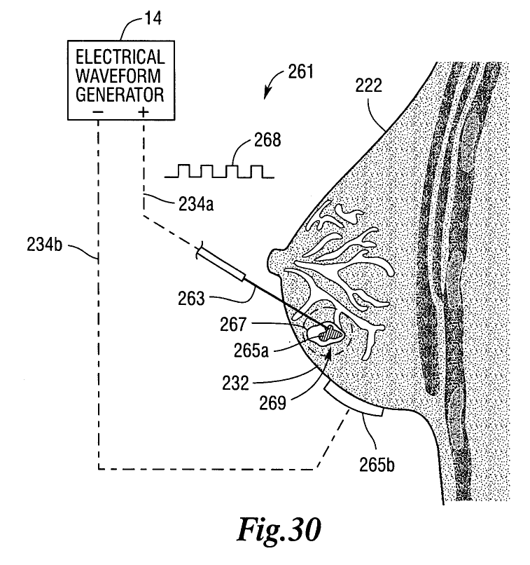

FIG. 30 illustrates one embodiment of an electrical ablation device 261 to treat diseased tissue within a breast by delivering electrical energy to a space defined within the breast. For example, the electrical ablation device 261 may be employed to treat breast cancer tissue in a target site 269 within a certain depth of a space 267 formed within a breast 222 defined by a lumpectomy procedure. A needle electrode 263 is located into the space 267 transcutaneously through the breast 222. The needle electrode 263 comprises an inflatable and deflatable balloon member 265a, or a sponge-type member, disposed at a distal end portion of the needle electrode 263. The balloon member 265a comprises at least one radially expandable hollow body. At least one electrode surface contact member is disposed at a peripheral portion of the hollow body. The needle electrode 263 is particularly suited for use in treating diseased tissue, such as cancerous tissue, located within a certain depth or margin into the breast 222 adjacent to or surrounding the space 267. The inflatable and deflatable balloon member 265a may be introduced into the space 267 through a central lumen defined in the needle electrode 263. The balloon member 265a is inflatable to form an electrode suitable to couple electrical fields to destroy tissue to a predetermined depth surrounding the space 267 in the target site 269, creating a margin. The balloon member 265a may be formed as a hollow body which may be inflated by a suitable liquid, such as a solution of NaCl, so as to expand radially into contact with the inner wall of the space 267. At the outer periphery of the hollow body there may be disposed a plurality of discrete electrode surface contact members, which may be evenly distributed around the circumference of the hollow body for making proper electrical contact with the inner wall of the space 267. The electrode surface contact members may be connected in parallel or individually to the electrical waveform generator 14 through a first lead 234a running internally or externally of the needle electrode 263.

A pad electrode 265b comprising an electrically conductive pad is located on an exterior or outside portion 232 of the breast 222. The pad electrode 265b has a much larger surface area that the balloon member 265a of the needle electrode 263. In one embodiment, the balloon member 265a of the needle electrode 263 and the pad electrode 265b are adapted to receive an electrical field generated by the electrical waveform generator 14. In one embodiment, the electrical field is in the form of an IRE waveform generated by an IRE generator. In another embodiment, the electrical field is in the form of a RF waveform generated by an RF generator. The needle electrode 263 is connected to the waveform generator 14 through a first lead 234a and the pad electrode 265b is connected to the waveform generator through a second lead 234b. In the illustrated embodiment, the needle electrode 263 is connected to a positive output of the waveform generator 14 and the pad electrode 265b is connected to a negative output of the waveform generator 14. As previously discussed, the electrical waveform generator 14 is capable of generating high voltage pulse waveforms of various amplitude, frequency, and pulse duration. In other embodiments, the polarity of the needle electrode 263 and the pad electrode 265b may be inverted. Multiple pulses may be supplied to the needle electrode 263 and the pad electrode 265b to destroy cancerous tissue at a certain depth of the space 267 near the target zone 269. A pulse train 268 comprising 20 to 40 pulses of ±500 to ±700 VDC of approximately 0.4 milliseconds in duration each is sufficient to destroy the cancerous tissue 226. As previously discussed, in one embodiment, the polarity of the electrical pulses may be inverted or reversed by the electrical waveform generator 14 during the treatment process.

The techniques discussed above with reference to FIGS. 23, 24, 25, and 30 also may be implemented to deliver RF energy to ablate of the cancerous tissue 226, or any electrical waveforms suitable to destroy diseased tissue cells at the treatment site.

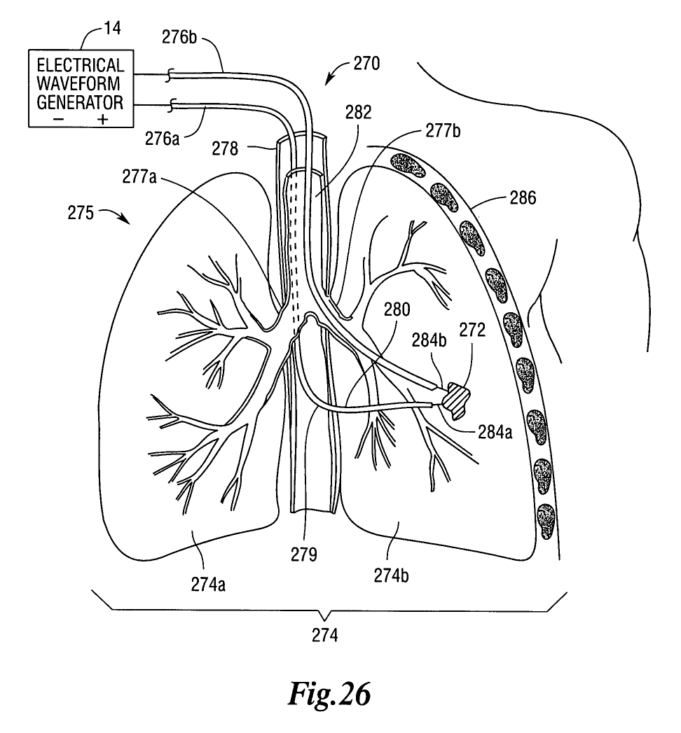

FIG. 26 illustrates one embodiment of an electrical ablation device 270 to treat diseased within a body cavity or organ by delivering electrical energy to the body cavity or organ. In the embodiment illustrated in FIG. 26, the electrical ablation device 270 is employed to treat tumors located in lungs 274. The embodiment, however, is not limited in this context and may be employed to treat tumors in any body cavity or organ. As illustrated in FIG. 26, the respiratory system 275 includes the trachea 282, which brings air from the nose or mouth into the right primary bronchus 277a and the left primary bronchus 277b. From the right primary bronchus 277a the air enters right lung 274a; from the left primary bronchus 277b the air enters the left lung 274b. The right lung 274a and the left lung 274b together form the lungs 274. The esophagus 278 extends into the thoracic cavity located behind the trachea 282 and the right and left primary bronchi 277a,b.

A lung tumor 272 is shown in the left lung 274b. The lung tumor 272 can be difficult to resect surgically. A first catheter 276a is introduced through a wall 279 of the esophagus 278, through lung tissue 280, and is located next to the tumor 272. A second catheter 276b is introduced through the trachea 282 and is located next to the tumor 272. The first and second catheters 276a,b are independently steerable. The first and second catheters 276a,b may be formed as hollow flexible tubes for insertion into a body cavity, duct, or vessel comprising first and second lumen to receive respective first and second elongated electrical conductors 284a,b therethrough. Each one of the first and second elongated electrical conductors 284a,b comprise a metal portion that extends beyond the distal end of the respective first and second catheters 276a,b. The proximal ends of the first and second electrical conductors 284a,b are coupled to the output electrodes of the waveform generator 14.

Electrical ablation by applying a suitable electrical field as discussed above is an effective way to destroy the lung tumor 272. In one embodiment, the first and second electrical conductors 284a,b are adapted to receive an IRE waveform from an IRE generator. In another embodiment, the first and second electrical conductors 284a,b are adapted to receive a RF waveform from an RF generator. Radio frequency ablation supplies energy into the cancerous tissue of the tumor 272 to raise its temperature and destroy the tumor 272. IRE employs high voltage DC pulses to destroy the tumor 272. The exposed metal portions of the electrical conductors 284a,b located within the respective first and second catheters 276a,b are located near the tumor 272 and high voltage DC pulses are applied to the cancerous tissue of the tumor 272 to destroy it. In one embodiment, the pulses may be extremely short in duration (˜5 microseconds) and may be applied in multiple bursts such as 20 to 40 pulses, for example. The voltage amplitude or energy of each pulse is sufficient to cause damage to the cells at the target site (e.g., cancerous tissue forming the tumor 272) by necrosis or inducing apoptosis, as discussed above. Both the first and second catheters 276a,b may be introduce through the esophagus 278, the trachea 282, the skin 286 or any combination thereof. As previously discussed, in one embodiment, the polarity of the electrical pulses may be inverted or reversed by the electrical waveform generator 14 during the treatment process.

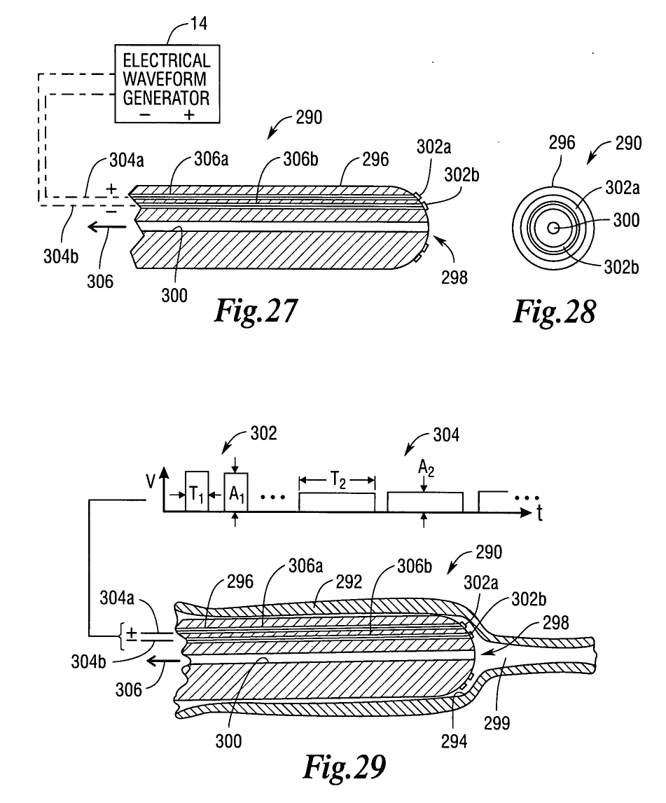

FIGS. 27, 28, and 29 illustrate one embodiment of an electrical ablation device 290 to treat diseased tissue within a body lumen using electrical energy. In the embodiment illustrated in FIGS. 27-29, the electrical ablation device is adapted to treat varicose veins. The embodiment, however, is not limited in this context. Reflux disease of the Greater Saphenous Vein (GSV) can result in a varicose vessel 292 as illustrated in FIG. 29. Conventional treatment techniques for varicose veins include stripping the vessel 292 and applying either chemical or thermal ablation to the vessel 292. The electrical ablation device 290 applies high voltage DC pulses to destroy a wall 294 of the vessel 292 and subsequently seal the vessel 292. FIG. 27 illustrates a sectioned view of one embodiment of an electrical ablation probe 296. FIG. 28 illustrates an end view of one embodiment of the electrical ablation probe 296. FIG. 29 is a cross-sectional view of one embodiment of the electrical ablation device 290 that may be inserted in a lumen 298 within the vessel or varicose vessel 292.

With reference to FIGS. 27-29, the probe 296 comprises a cannula or lumen 300 extending longitudinally therethrough. The distal end 298 the probe 296 comprises first and second ring electrodes 302a,b at a potential difference. The first and second ring electrodes 300a,b are coupled to positive and negative electrodes or terminals of the electrical waveform generator 14 through first and second conductors 304a,b extending through respective conduits 306a,b formed within the probe 296 and extending longitudinally therethrough. The first and second conductors 304a,b may be electrically coupled to the first and second ring electrodes 302a,b in any suitable manner. The first and second ring electrodes 302a,b are adapted to receive an electrical field from a suitable generator. In one embodiment, the first and second ring electrodes 302a,b are adapted to receive an electrical field from a generator such as IRE waveform from an IRE generator. In another embodiment, the first and second ring electrodes 302a,b are adapted to receive an electrical field from a generator such as a RF waveform from an RF generator.

The electrical ablation probe 296 has a form factor that is suitable to be located into a tapered lumen 298 of the vessel 292. The probe 296 engages the vessel wall 294 as it is inserted within the tapered lumen 299 of the vessel 292. Suction 306 applied at a proximal end of the probe 296 draws a vacuum within the lumen 300 of the probe causing the vessel 292 to collapse at the distal end 298 of the probe 296.

Once the vessel 292 is collapsed or pulled down by the suction 306, a first pulse train 302 comprising high voltage DC pulses of a first amplitude A₁ (e.g., ˜1 KV amplitude) and a first pulse duration T₁ (e.g., ˜50 microseconds) is applied to the first and second ring electrodes 300a,b by the electrical waveform generator 14. The high voltage DC pulse train 302 eventually causes the cells to die. A second pulse train 304 having a lower voltage amplitude A₂ (e.g., −500VDC) and a second pulse duration T₂ (e.g., ˜15 milliseconds) is applied to the first and second ring electrodes 300a,b of the probe 296 to cause thermal damage and seal the vein 292. As previously discussed, in one embodiment, the polarity of the electrical pulses may be inverted or reversed by the electrical waveform generator 14 during the treatment process.

The devices disclosed herein can be designed to be disposed of after a single use, or they can be designed to be used multiple times. In either case, however, the device can be reconditioned for reuse after at least one use. Reconditioning can include any combination of the steps of disassembly of the device, followed by cleaning or replacement of particular pieces, and subsequent reassembly. In particular, the device can be disassembled, and any number of the particular pieces or parts of the device can be selectively replaced or removed in any combination. Upon cleaning and/or replacement of particular parts, the device can be reassembled for subsequent use either at a reconditioning facility, or by a surgical team immediately prior to a surgical procedure. Those skilled in the art will appreciate that reconditioning of a device can utilize a variety of techniques for disassembly, cleaning/replacement, and reassembly. Use of such techniques, and the resulting reconditioned device, are all within the scope of the present application.

Preferably, the various embodiments of the invention described herein will be processed before surgery. First, a new or used instrument is obtained and if necessary cleaned. The instrument can then be sterilized. In one sterilization technique, the instrument is placed in a closed and sealed container, such as a plastic or TYVEK® bag. The container and instrument are then placed in a field of radiation that can penetrate the container, such as gamma radiation, x-rays, or high-energy electrons. The radiation kills bacteria on the instrument and in the container. The sterilized instrument can then be stored in the sterile container. The sealed container keeps the instrument sterile until it is opened in the medical facility.

It is preferred that the device is sterilized. This can be done by any number of ways known to those skilled in the art including beta or gamma radiation, ethylene oxide, steam.

Although the various embodiments of the invention have been described herein in connection with certain disclosed embodiments, many modifications and variations to those embodiments may be implemented. For example, different types of end effectors may be employed. Also, where materials are disclosed for certain components, other materials may be used. The foregoing description and following claims are intended to cover all such modification and variations.

Any patent, publication, or other disclosure material, in whole or in part, that is said to be incorporated by reference herein is incorporated herein only to the extent that the incorporated materials does not conflict with existing definitions, statements, or other disclosure material set forth in this disclosure. As such, and to the extent necessary, the disclosure as explicitly set forth herein supersedes any conflicting material incorporated herein by reference. Any material, or portion thereof, that is said to be incorporated by reference herein, but which conflicts with existing definitions, statements, or other disclosure material set forth herein will only be incorporated to the extent that no conflict arises between that incorporated material and the existing disclosure material.

What is claimed is:

1. An ablation device comprising:
an elongated flexible member having a proximal end and a distal end, the flexible member comprising first and second lumen;
a first needle electrode configured to slideably move within the first lumen; and
a second needle electrode located within the second lumen;
wherein the first and second needle electrodes are adapted to couple to an electrical waveform generator and to receive an electrical waveform sufficient to electrically ablate tissue located between the first and second needle electrodes.

2. The ablation device of claim 1, wherein the electrical waveform comprises a first pulse having a magnitude, polarity, and duration suitable to irreversibly destroy tissue cells located between the first and second needle electrodes.

3. The ablation device of claim 2, wherein the electrical waveform comprises a second pulse having a polarity that is the reverse polarity of the least first pulse.

4. The ablation device of claim 1, comprising:
a handle portion to receive a proximal end of the elongated flexible member;
a first slide member coupled to the first needle electrode to slideably advance or retract the first needle electrode; and
first and second receptacles to receive electrical conductive elements to couple the first and second needle electrodes to the electrical waveform generator
wherein the second needle electrode is slideably moveable within the second lumen and the ablation device comprises a second slide member coupled to the second needle electrode to slideably advance or retract the second needle electrode;

5. The ablation device of claim 4, comprising:
a sensor located within the handle portion to sense the force with which at least one of the first and second needle electrodes penetrate tissue in a tissue treatment zone;

6. An ablation device comprising:
an elongated flexible member having a proximal end and a distal end, the flexible member comprising at least one lumen;
a clevis coupled to the elongated flexible member;
first and second jaw members pivotally coupled to the clevis forming a clamp jaw, the first and second jaw members comprising respective first and second electrodes to couple to an electrical waveform generator; and
an elongated actuator member slidably received within the at least one lumen, the elongated actuator member coupled to the clevis, wherein longitudinal motion of the elongated actuator element in a first longitudinal direction opens the first and second jaw members and longitudinal motion in a second opposite direction closes the first and second jaw members;
wherein the first and second electrodes are adapted to couple to an electrical waveform generator and to receive an electrical waveform sufficient to electrically ablate tissue located between the first and second jaw members.

7. The ablation device of claim 6, wherein the electrical waveform comprises a first pulse having a magnitude, polarity, and duration suitable to irreversibly destroy tissue cells located between the first and second needle electrodes.

8. The ablation device of claim 7, wherein the electrical waveform comprises a second pulse having a polarity that is the reverse polarity of the least first pulse.

9. The ablation device of claim 6, comprising:
a handle portion to receive a proximal end of the elongated actuator member; and
a trigger operatively couple to the elongated actuator member;
wherein when the trigger is pivotally moved in a first rotational direction the elongated actuator member moves in the first longitudinal direction to open the first and second jaw members and when the trigger is pivotally moved in a second rotational direction the elongated actuator member moves in the second longitudinal direction to close the first and second jaw members.

10. The ablation device of claim 6, comprising:
an actuator coupled to the elongated actuator member and coupled to the first and second jaw members; and
a bushing coupler and ring capture coupled to the clevis and coupled to the elongated flexible member.

11. An ablation device comprising:
a tubular member comprising a body defining a central opening to receive a flowable substance; and
first and second electrodes formed on the tubular member to couple to an electrical waveform generator;
wherein the first and second electrodes are adapted to couple to an electrical waveform generator and to receive an electrical waveform sufficient to electrically treat the flowable substance flowing through the central opening.

12. The ablation device of claim 11, wherein the electrical waveform comprises a first pulse having a magnitude, polarity, and duration suitable to irreversibly destroy tissue cells located between the first and second needle electrodes.

13. The ablation device of claim 12, wherein the electrical waveform comprises a second pulse having a polarity that is the reverse polarity of the least first pulse.

14. The ablation device of claim 11, wherein the body is expandable.

15. The ablation device of claim 11, comprising:
first and second ring electrodes formed at a distal end of the tubular member;
at least one lumen to receive first and second conductors, the conductors are coupled to the respective first and second ring electrodes;
wherein the first and second ring electrodes are adapted to couple to an electrical waveform generator and to receive an electrical waveform sufficient to treat diseased tissue within a body lumen.

16. The ablation device of claim 11, wherein the central opening is adapted to couple to a vacuum device;

17. An ablation device, comprising:
a first electrode comprising an electrically conductive elongated member to be received within a breast; and
wherein the first electrode is adapted to couple to an electrical waveform generator and to receive an electrical waveform sufficient to electrically treat diseased tissue occupying the lactiferous duct.

18. The ablation device of claim 17, wherein the electrical waveform comprises a first pulse having a magnitude, polarity, and duration suitable to irreversibly destroy tissue cells located between the first and second needle electrodes.

19. The ablation device of claim 18, wherein the electrical waveform comprises a second pulse having a polarity that is the reverse polarity of the least first pulse.

20. The ablation device of claim 17, comprising:
a second electrode comprising electrically conductive elongated member to be received within a breast.

21. The ablation device of claim 17, comprising an electrically conductive pad to be located on an exterior portion of the breast.

22. An electrical ablation device, comprising:
first and second steerable catheters defining respective first and second lumen therein; and
first and second electrical conductors located within the respective first and second lumen, a portion of the first and second electrical conductors extend beyond a distal end of the respective first and second steerable catheters;
wherein the first and second electrodes are adapted to couple to an electrical waveform generator and to receive an electrical waveform sufficient to electrically treat diseased tissue within a body cavity or organ.

23. The ablation device of claim 22, wherein the electrical waveform comprises a first pulse having a magnitude, polarity, and duration suitable to irreversibly destroy tissue cells located between the first and second needle electrodes.

24. The ablation device of claim 23, wherein the electrical waveform comprises a second pulse having a polarity that is the reverse polarity of the least first pulse.

25. A method of preparing an instrument for surgery, comprising:
obtaining the device of claim 1;
sterilizing the surgical instrument; and
storing the surgical instrument in a sterile container.
